(19) United States
(12) Patent Application Publication
(10) Pub. No.: US 2026/0146939 A1
(43) Pub. Date: May 28, 2026

(54) DETECTING CANCER

(71) Applicant: UCL BUSINESS LTD, London (GB)

(72) Inventors: Samuel GAMBLE, London (GB); James READING, London (GB); Sam JANES, London (GB); Maxine TRAN, London (GB); Vitor TEIXEIRA, London (GB); Claudia PEINADOR, London (GB)

(21) Appl. No.: 19/115,601

(22) PCT Filed: Sep. 26, 2023
(86) PCT No.: PCT/EP2023/076511

§ 371 Date: Mar. 26, 2025

(30) Foreign Application Priority Data
Sep. 27, 2022 (GB) 2214114.7
Sep. 27, 2022 (GB) 2214117.0

(51) Int. Cl. G01N 15/14; C12Q 1/34; G01N 15/10; G01N 33/68

(52) U.S. Cl. CPC G01N 15/14; C12Q 1/34; G01N 33/6872; G01N 2015/1006; G01N 2015/1486; G01N 2015/1488; G01N 2333/4703; G01N 2333/914

(57) ABSTRACT

A method for determining whether a subject is at risk for having a progressing or high-grade pre-invasive lesion, nodule or small mass, or having a solid malignant tumour is described the method comprising: (i) determining a ratio of activated and/or exhausted T cells:naive and/or resting T cells in a sample of blood obtained from the subject, wherein the determining comprises analysing T cells using cytometry to detect the presence or absence of a panel of biomarkers comprising Ki67 and CD39, or (ii) determining a ratio of activated and/or exhausted T cells:T cells which are not activated and/or exhausted T cells in a sample of blood obtained from the subject, wherein the determining comprises analysing T cells using cytometry to detect the presence or absence of a panel of biomarkers comprising Ki67 and CD39, (iii) determining a proportion of activated and/or exhausted T cells as a percentage of T cells in a sample of blood obtained from the subject, wherein the determining comprises analysing T cells using cytometry to detect the presence of a panel of biomarkers comprising Ki67 and CD39, and/or (iv) determining a proportion of activated and/or exhausted T cells as a percentage of T cells in a sample of blood obtained from the subject, wherein the T cells are CD4 T cells, and wherein the determining comprises analysing T cells using cytometry to detect the presence of a panel of biomarkers comprising FoxP3.

### BACKGROUND

Cancer is a leading cause of disease worldwide. Certain types of cancer have a high chance of cure if they are detected at an early stage and adequately treated. However, many cancers are detected at a late stage and delays in cancer diagnosis can occur throughout the diagnostic pathway. This can include patients failing to recognise symptoms or delaying see a healthcare provider. Doctors may not recognise symptoms of cancers and so may not investigate them appropriately or refer on time. Furthermore, some cancers are difficult to detect. Late diagnosis is a major cause of cancer mortality.

Renal cell carcinoma (RCC) is the cause of approximately 5,000 cancer deaths in the UK per year and non-small cell lung cancer (NSCLC) causes approximately 35,000 cancer deaths in the UK per year, with a combined annual NHS cost of over £3bn. This human and financial cost is replicated around the world. More than 40% of NSCLC and 10-20% of RCC cases are diagnosed late, representing a major cause of mortality. More than 40% of patients diagnosed with non-small cell lung cancer (NSCLC) present with late-stage disease (Stage 3-4), when the cancer has spread and where 5-year survival rates are dismal (90% mortality). As a result, NSCLC is the leading cause of cancer death both worldwide and in the UK, where the disease causes over 20% of the 160′000 annual cancer deaths. In addition, NSCLC incurs significant resource burden(s) costing the NHS an estimated £2.4bn each year and a global cost estimated to exceed £150bn. Patients diagnosed with Stage 1 NSCLC are ten times more likely to survive 5 years than those diagnosed at stage 4, engendering efforts for early detection of NSCLC.

NSCLC is comprised of two major histological subtypes;
i) lung adenocarcinoma (LUAD), which is localised to the lung parenchyma in the periphery and accounts for approximately 60-65% of disease and
ii) lung squamous cell carcinoma (LUSC) is found in the central airways, which constitutes approximately 30% of disease.

Each subtype has distinct forms of pre-malignant disease due to unique clinical and genomic features meaning that LUAD and LUSC are preferentially detected via specific screening approaches.

Low-dose computerised tomography (LDCT) lung screening is advocated as a major pathway for early lung cancer detection and predominantly detects LUAD. This strategy increases early diagnosis and reduces mortality by 20-26% but even the perfectly executed CT screening programme will only prevent 20% of deaths, as it targets the highest-risk patients, missing lighter and never smokers. Additional disadvantages of LDCT include resource intensiveness, radiation exposure and failure to detect most LUSCs. LDCT also detects pre-malignant and benign peripheral lung nodules, but these have indeterminate disease penetrance and require continual surveillance.

The pre-invasive stages of LUSC (‘pre-LUSC’) are characterised by asymptomatic lesions that develop in a stepwise process of increasing dysplasia and can be classified as low-grade (LG) and high-grade (HG). LG lesions are associated with no increased risk of NSCLC but 40-90% of patients with HG lesions progress. Traditional screening of pre-LUSC relies on sputum cytology which has poor sensitivity, and autofluorescence bronchoscopy which suffers from high false-positive rates, low patient throughput and the burden(s) associated with an invasive procedure. Similarly, whilst a 280-gene classifier of airway bronchial cells has been successfully used to predict the presence of pre-LUSC lesions this remains an expensive and invasive approach.

New non-invasive, tools to detect progressing lesions/nodules would address a vast clinical unmet need. Circulating tumour DNA (ctDNA) is the most widely proposed future clinical strategy for liquid multi-cancer detection but detects only 40-50% of stage I-II NSCLC cancers, falling to as low as 10% for stage I LUAD and is expected to be less effective in the pre-invasive setting.

Thus, current NSCLC detection protocols are often insensitive, non-specific, time-consuming, resource-heavy, invasive/painful and can lead to anxiety and over diagnosis or delayed diagnosis.

It is known that T cell differentiation is skewed in subjects with established or late stage cancer. The balance of T cell populations in a subject shifts towards the majority of T cells exhibiting biomarkers that are associated with having identified their cognate antigen on a tumour.

However, there is still no means for early cancer detection at the stage detecting a progressing or high-grade pre-invasive lesion or nodule or solid tumours before the cancer is established in the subject.

There therefore remains an unmet and urgent need for early detection of NSCLC and other solid cancers. This need is noted by Cancer Research UK (CRUK), the NHS long-term plan, the Medical Research Council (MRC) and the UK Research Institution (UKRI).

The ability to distinguish between subjects with early-stage solid cancers or pre-invasive lesions or nodules likely to progress to cancer, versus subjects with no malignancy or with lesions or nodules unlikely to progress would enable early, curative intervention or targeted surveillance, saving lives and significantly reducing resource burden.

### THE PRESENT INVENTION

The present invention aims to solve these and other problems by providing a novel method for determining whether a subject is at risk for having a progressing or high-grade pre-invasive lesion or nodule. The present invention can also determine whether a subject is at risk for having a solid malignant tumour of any stage. The present invention provides, for the first time, a non-invasive immune-based method for distinguishing between low-risk pre-invasive lesions or nodules, low grade pre-invasive lesions or nodules (or lack of tumour), and progressing pre-invasive lesions or nodules, high grade pre-invasive lesions or nodules (or having a solid tumour, e.g., a stage I solid tumour). The present invention also detects pre-invasive lesions or nodules (e.g., high grade pre-invasive lesions or nodules) that are at risk of progressing via measuring immunological markers in the blood, and provides a readout as compared with a healthy subject or a plurality of healthy subjects.

The use of biomarkers to determine a subject's T cell differentiation state in a blood sample obtained from a subject, that can be used in the early detection setting to detect the presence of progressive pre-invasive lesions or nodules, or early-stage tumours is described herein for the first time. At the time of writing, all prior work with T cell biomarkers only related to established late-stage cancer (i.e., stage III or IV cancer). It was not known that T cell biomarkers could be detectable prior to established cancer at the pre-invasive stage, or in early-stage cancer (e.g., stage I cancer).

When T cells recognise non-self antigen they differentiate from a naïve or resting state into a range of activated, exhausted and memory T cells. These changes can be measured by cytometry. Cytometry in this context, is used to profile the frequency and intensity of biomarkers on T cells. The biomarkers to be detected can be those related to T cell activation exhaustion and memory differentiation. Combinations of these biomarkers can be used to determine the strength or type of immune activation in disease states such as infection, autoimmunity, transplantation and cancer. These biomarkers can be measured within CD3+ live cells in the blood (viable T cells) and amongst the major T cell lineages of killer cytotoxic T cells (‘CD8+ T cells’) or helper & regulatory (‘CD4 T cells’).

In chronic infections like HIV-1 and HCV there is well established expansion of activated, memory and exhausted T cells at the expense of resting and naïve T cells in the blood. The level of activated and exhausted T cells reflects the presence of disease compared to healthy individuals and the amount of virus detectable. This shift in differentiation is termed T cell differentiation skewing. It has been discovered that a similar remodelling of T cell differentiation inside the tumours of patients with established NSCLC and RCC; is characterised by T cells co-expressing biomarkers of exhaustion activation and terminal differentiation and loss of stem-like populations expressing biomarkers of early-differentiation and progenitor potential.

T cell differentiation skewing in the blood of cancer patients is less well characterised.

The present inventors have shown in NSCLC, that (neo)antigen load (inferred by tumour mutational burden) correlated with T cell differentiation skewing inside the tumour (loss in resting, progenitor and naïve cells, increase in terminally differentiated, exhausted and activated T cells).

The use of T cell differentiation skewing, defined as a loss of resting/naïve T cells and a gain in activated/exhausted T cells as a method of early cancer detection, such as early lung cancer detection, has not previously been described. Prior to the present invention there is no published data to show that the presence of pre-invasive LUSC lesions or pre-LUAD nodules are associated with blood T cell differentiation skewing. Equally prior to the present invention there was no data to support an equivalent process in another solid tumour type.

Prior to the present invention, existing methods and strategies, such as disclosed in US2018356420 A1 have focused on using antibody panels to analyse T cells within patients with established cancer. However, these methods have limitations as they cannot be used for distinguishing between progressing and non-progressing lesions or nodules, nor for determining whether a subject is at risk for having a progressing or high-grade pre-invasive lesion or nodule or having a very early stage (stage 1) solid tumour.

Elsewhere in earlier studies, Li et al, Lung Cancer 162 (2021) 16-22 describes lung cancer-associated T cell repertoire as potential biomarker for detection of stage lung cancer. The authors do not propose detection of lesions or nodules before a cancer is established. The authors do not propose analysis of the T cell repertoire for distinguishing between progressing and non-progressing lesions or nodules, nor for determining whether a subject is at risk for having a progressing or high-grade pre-invasive lesion or nodule. The authors do not use the ratio of exhausted/activated to naïve/resting T cells as a method for early stage cancer detection.

Mascaux et al, 570, Nature, Vol 571, 25 Jul. 2019, relates to immune evasion before tumour invasion in early lung squamous carcinogenesis and indicates that the adaptive immune response within tumours may be strongest at the earliest stage of carcinoma. However, there is no suggestion of distinguishing between progressing and non-progressing lesions or nodules, nor determining whether a subject is at risk for having a progressing or high-grade pre-invasive lesion or nodule or having a solid tumour.

WO2021188941 A1 relates to methods for isolating T cells and T-cells receptors from peripheral blood by single-cell analysis for immunotherapy, in addition to preparing and enriching a population of T cells having antigenic specificity for a target antigen. However, the authors focused on analysing and using the T cell repertoire as a therapeutic strategy for established cancer in patients and does not propose a method for distinguishing between progressing and non-progressing lesions or nodules, nor for determining whether a subject is at risk for having a progressing or high-grade pre-invasive lesion or nodule or having a solid tumour.

Cancers, vol. 14, 2022, “Martinez-Gomez et al” identifies certain T cell biomarkers as markers for tumor specific T cells or T cells which are a surrogate for an active immune response. However, this work focused on samples with established disease, with no suggestion or teaching of what biomarkers, if any, could be used for pre-invasive or early stage (e.g., stage I) disease.

Immune Network, vol 20(6), 2020, “Kim et al” article e48, similarly focuses on T cells in cancer patients, and more specifically highlight the difference between those that do or do not respond to checkpoint inhibitors with clinical benefit. However, this document also only relates to later stage disease and response to a therapy. This does not show any patients with preinvasive or early-stage disease who are distinguished by these cells compared with no disease.

### STATEMENTS OF THE INVENTION

In a first aspect, the present invention provides a method for determining whether a subject is at risk for having a progressing or high-grade pre-invasive lesion, nodule or small mass, or having a solid malignant tumour, the method comprising:
(i) determining a ratio of activated and/or exhausted T cells:naïve and/or resting T cells in a sample of blood obtained from the subject, wherein the determining comprises analysing T cells using cytometry to detect the presence or absence of a panel of biomarkers comprising Ki67 and CD39, or
(ii) determining a ratio of activated and/or exhausted T cells:T cells which are not activated and/or exhausted T cells in a sample of blood obtained from the subject, wherein the determining comprises analysing T cells using cytometry to detect the presence or absence of a panel of biomarkers comprising Ki67 and CD39,
(iii) determining a proportion of activated and/or exhausted T cells as a percentage of T cells in a sample of blood obtained from the subject, wherein the determining comprises analysing T cells using cytometry to detect the presence of a panel of biomarkers comprising Ki67 and CD39 and/or
(iv) determining a proportion of activated and/or exhausted T cells as a percentage of T cells in a sample of blood obtained from the subject, wherein the T cells are CD4 T cells, and wherein the determining comprises analysing T cells using cytometry to detect the presence of a panel of biomarkers comprising FoxP3.

In a second aspect or embodiment of the first aspect, the present invention provides a method for determining whether a subject is at risk for having a progressing or high-grade pre-invasive lesion, nodule or small mass, or having a solid malignant tumour, the method comprising:
determining a ratio of activated and/or exhausted T cells:naïve and/or resting T cells in a sample of blood obtained from the subject,
wherein the determining comprises analysing T cells using cytometry to detect the presence or absence of a panel of biomarkers comprising Ki67 and CD39. In some embodiments, the solid malignant tumour is a stage I malignant tumour.

The subject is at risk if
the ratio of activated and/or exhausted T cells:naïve and/or resting T cells is equal to or greater than
a ratio of activated and/or exhausted T cells:naïve and/or resting T cells of a comparison subject, or
an average ratio of activated and/or exhausted T cells:naïve and/or resting T cells of a plurality of comparison subjects.

In a third aspect or embodiment of the first aspect, the present invention also provides a method for determining whether a subject is at risk for having a progressing or high-grade pre-invasive lesion, nodule or small mass, or having a solid malignant tumour, the method comprising:
determining a ratio of activated and/or exhausted T cells:T cells which are not activated and/or exhausted T cells in a sample of blood obtained from the subject, wherein the determining comprises analysing T cells using cytometry to detect the presence or absence of a panel of biomarkers comprising Ki67 and CD39. In some embodiments, the solid malignant tumour is a stage I malignant tumour.

The subject is at risk if
the ratio of activated and/or exhausted T cells:T cells which are not activated and/or exhausted is equal to or greater than
a ratio of activated and/or exhausted T cells:T cells which are not activated and/or exhausted of a comparison subject, or
average ratio of activated and/or exhausted T cells:T cells which are not activated and/or exhausted of a plurality of comparison subjects.

In a fourth aspect or embodiment of the first aspect, the present invention also provides a method for determining whether a subject is at risk for having a progressing or high-grade pre-invasive lesion, nodule or small mass, or having a solid malignant tumour, the method comprising:
determining a proportion of activated and/or exhausted T cells as a percentage of T cells in a sample of blood obtained from the subject, wherein the determining comprises analysing T cells using cytometry to detect the presence of a panel of biomarkers comprising Ki67 and CD39. In some embodiments, the solid malignant tumour is a stage I malignant tumour.

The subject is as risk if
the proportion of activated and/or exhausted T cells as a percentage of T cells is greater than the proportion of activated and/or exhausted T cells as a percentage of T cells in a comparison subject or
the proportion of activated and/or exhausted T cells as a percentage of total T cells is greater than the average proportion of activated and/or exhausted T cells as a percentage of T cells in a plurality of comparison subjects.

For all of the above aspects and embodiments, a comparison subject is selected from:
a) a subject known to have a progressing or high-grade pre-invasive lesion, nodule or small mass or an established solid malignant tumour,
b) the subject at a different time point.

A plurality of comparison subjects is selected from:
a) a plurality of subjects known to have a progressing or high-grade pre-invasive lesion, nodule or small mass or an established solid malignant tumour,
b) a plurality of healthy subjects, or
c) a plurality of subjects of the general population.

For the second, third and fourth aspects (and in embodiments of the first aspect):
An activated and/or exhausted T cell expresses CD39 and Ki67 (CD39+ and Ki67+ T cells).
A naïve and/or resting T cell does not express CD39 or Ki67 (CD39− and Ki67− T cells).
A T cell which is not an activated and/or exhausted does not express one or both of CD39 or Ki67 (CD39− and Ki67+ T cells, CD39+ and Ki67− T cells, or CD39− and Ki67− T cells).
In some embodiments, the panel of biomarkers may further comprise one or more biomarkers selected from CD45RA, CCR7, PD-1, CD57 or CD38.
In some embodiments, the panel may further comprise one or more biomarkers selected from CD45RA, CCR7, PD-1, CD57, CD38 or FoxP3.
In some embodiments, the panel of biomarkers may further comprise one or more biomarkers selected from CD3, CD4 or CD8.
In some embodiments, the analysis may also comprise use of a viability dye.
In some embodiments, the panel of biomarkers may further comprise CD45RA, or CCR7, or CD45RA and CCR7.
In some embodiments, the panel of biomarkers may further comprise CD45RA, or CCR7, or FoxP3, or CD45RA and CCR7.
In some embodiments, the panel of biomarkers may further comprise CD45RA, CCR7, and PD-1.
In some embodiments, the panel of biomarkers may further comprise CD45RA, CCR7, PD-1 and FoxP3.
In some embodiments, the panel of biomarkers may further comprise CD45RA, CCR7, PD-1 and CD57.
In some embodiments, the panel of biomarkers may further comprise CD45RA, CCR7, PD-1, CD57 and FoxP3.
In some embodiments, the panel of biomarkers may further comprise CD45RA, CCR7, PD-1, CD57 and CD38.
In some embodiments, the panel of biomarkers may further comprise CD45RA, CCR7, PD-1, CD57, CD38 and FoxP3.
In some embodiments, the panel of biomarkers may further comprise CD45RA, CCR7, CD57, and CD38.
In some embodiments, the panel of biomarkers may further comprise CD45RA, CCR7, CD57, CD38 and FoxP3.
In some embodiments, the panel of biomarkers may further comprise CD45RA, PD-1 and CD57.
In some embodiments, the panel of biomarkers may further comprise CD45RA, PD-1, CD57 and FoxP3.
In some embodiments, the panel of biomarkers comprises Ki67, CD39, CCR7, PD1. In some embodiments, the panel of biomarkers comprises Ki67, CD39, CCR7, PD1 and CD8. In some embodiments, the panel of biomarkers comprises Ki67, CD39, CCR7, PD1, CD8 and CD3. In such embodiments, the activated and/or exhausted cell expresses Ki67 and CD39 and does not express CD45RA, CCR7 or PD1.

In a fifth aspect of the present invention, and in an embodiment of the first aspect, the present invention also provides a method for determining whether a subject is at risk for having a progressing or high-grade pre-invasive lesion, nodule or small mass, or having a solid malignant tumour, the method comprising:
determining a proportion of activated and/or exhausted CD4 T cells as a percentage of CD4 T cells in a sample of blood obtained from the subject, wherein the determining comprises analysing T cells using cytometry to detect the presence of a panel of biomarkers comprising FoxP3.

The subject is as risk if
the proportion of activated and/or exhausted CD4 T cells as a percentage of CD4 T cells is greater than the proportion of activated and/or exhausted T cells as a percentage of CD4 T cells in a comparison subject or
the proportion of activated and/or exhausted T cells as a percentage of total CD4 T cells is greater than the average proportion of activated and/or exhausted T cells as a percentage of CD4 T cells in a plurality of comparison subjects.

A comparison subject is selected from:
a) a subject known to have a progressing or high-grade pre-invasive lesion, nodule or small mass or an established solid malignant tumour,
b) the subject at a different time point.

A plurality of comparison subjects is selected from:
a) a plurality of subjects known to have a progressing or high-grade pre-invasive lesion, nodule or small mass or an established solid malignant tumour,
b) a plurality of healthy subjects, or
c) a plurality of subjects of the general population.

In the fifth aspect (and in embodiments of the first aspect)
The activated and/or exhausted T cell is a CD4 T cell that expresses FoxP3. The panel of biomarkers comprises FoxP3 (and optionally CD4). The panel of biomarkers may further comprise one or more biomarkers selected from CD39, Ki67, CD45RA, CCR7, PD-1, CD57 or CD38.
In some embodiments, the panel of biomarkers may comprise FoxP3, CD39 (and optionally CD4). In some embodiments, the activated and/or exhausted T cell is a CD4 T cell that expresses FoxP3 in combination with CD39.
In some embodiments, the panel of biomarkers may comprise FoxP3, CD39 and Ki67 (and optionally CD4). In some embodiments, the activated and/or exhausted T cell is a CD4 T cell that expresses FoxP3, in combination with CD39 and Ki67. In some embodiments, the panel of biomarkers may further comprise one or more biomarkers selected from CD45RA, CCR7, PD-1, CD57 or CD38. In some embodiments, the panel of biomarkers may further comprise CD45RA, or CCR7, or CD45RA and CCR7. In some embodiments, the panel of biomarkers may further comprise CD45RA, CCR7, and PD-1. In some embodiments, the panel of biomarkers may further comprise CD45RA, CCR7, PD-1 and CD57. In some embodiments, the panel of biomarkers may further comprise CD45RA, CCR7, PD-1, CD57 and CD38. In some embodiments, the panel of biomarkers may further comprise CD45RA, CCR7, CD57, and CD38. In some embodiments, the panel of biomarkers may further comprise CD45RA, PD-1 and CD57. In some embodiments, the panel of biomarkers may further comprise a CD3 biomarker. In some embodiments, the analysis may also comprise use of a viability dye.
In some embodiments, the panel of biomarkers may comprise FoxP3, CD39 and CD45RA (and optionally CD4). The activated and/or exhausted T cell is a CD4 T cell that expresses FoxP3 in combination with CD39, but which does not express CD45RA. In some embodiments, the panel of biomarkers may further comprise one or more biomarkers selected from CCR7, PD-1, CD57 or CD38. In some embodiments, the panel of biomarkers may further comprise CCR7. In some embodiments, the panel of biomarkers may further comprise CCR7 and PD-1. In some embodiments, the panel of biomarkers may further comprise CCR7, PD-1 and CD57. In some embodiments, the panel of biomarkers may further comprise CCR7, PD-1, CD57 and CD38. In some embodiments, the panel of biomarkers may further comprise PD-1 and CD57. In some embodiments, the panel of biomarkers may further comprise a CD3 biomarker. In some embodiments, the analysis may also comprise use of a viability dye.

The fifth aspect of the present invention comprises determining a proportion of activated and/or exhausted CD4 T cells as a percentage of CD4 T cells in a sample of blood obtained from the subject, wherein the determining comprises analysing T cells using cytometry to detect the presence of a panel of biomarkers comprising FoxP3.

This method may encompass or may be used interchangeably with a method that comprises determining a proportion of CD4 T regulatory cells as a percentage of CD4 T cells in a sample of blood obtained from the subject wherein the determining comprises analysing T cells using cytometry to detect the presence of a panel of biomarkers comprising FoxP3. CD4 T regulatory cells/CD4+T regulatory cells (also disclosed as T_{reg} herein) express the biomarkers FoxP3 and CD4. In some embodiments, the CD4+T regulatory cells express FoxP3, CD39 and CD4. In some embodiments, the CD4+T regulatory cells are CD4 Proliferating regulatory T cells (also disclosed as Treg.prolif herein). CD4 Proliferating regulatory T cells express FoxP3, CD4 and CD39 in combination with Ki67. CD4 Proliferating regulatory T cells may express FoxP3, Ki67, CD4 and CD39, but not express one or more of CD45RA and CCR7. In some embodiments, the CD4+T regulatory cells express FoxP3, CD39 and CD4, but may not express one or more of Ki67, CD45RA or CCR7, or a combination of Ki67, CD45RA and CCR7.

The method may encompass or may be used interchangeably with a method that comprises determining a proportion of effector regulatory T cells in a sample of blood from the subject, wherein the determining comprises analysing T cells using cytometry to detect the presence of a panel of biomarkers comprising FoxP3.

For all the above aspects and embodiments, cytometry comprises one or more of flow cytometry, spectral cytometry or mass cytometry. Optionally the cytometry comprises flow cytometry.

The invention also provides a kit comprising a set of lyophilised antibodies or fragment thereof, comprising (i) antibodies binding to CD39 and Ki67 or (ii) antibodies binding to FoxP3, or FoxP3 and CD4, optionally in combination with an antibody binding to CD39.

The invention also provides a device comprising:
(i) means for receiving a sample of blood, and
(ii) a set of lyophilised antibodies or a fragment thereof, comprising antibodies binding to (i) CD39 and Ki67 and/or (ii) antibodies binding to FoxP3, or FoxP3 and CD4, optionally in combination with an antibody binding to CD39

A set of lyophilised antibodies or fragment thereof may comprise antibodies binding to the further biomarkers:
CD45RA, or CCR7, or CD45RA and CCR7.
CD45RA and FoxP3, CCR7 and FoxP3, or CD45RA, CCR7 and FoxP3.
CD45RA, CCR7, and PD-1.
CD45RA, CCR7, PD-1 and FoxP3.
CD45RA, CCR7, PD-1 and CD57.
CD45RA, CCR7, PD-1, CD57 and FoxP3.
CD45RA, CCR7, PD-1, CD57 and CD38.
CD45RA, CCR7, PD-1, CD57, CD38 and FoxP3.
CD45RA, CCR7, CD57, and CD38.
CD45RA, CCR7, CD57, CD38, and FoxP3.
CD45RA, PD-1 and CD57.
CD45RA, PD-1, CD57 and FoxP3.

The invention further provides use of the kit described above in a method for determining whether a subject is at risk for having a progressing or high-grade pre-invasive lesion or nodule or having a solid tumour.

This invention further provides a method of treating a subject determined to be at risk for having a progressing or high-grade pre-invasive lesion, nodule or small mass, or having a solid malignant tumour, wherein the method comprises:
determining whether a subject is at risk for having a progressing or high-grade pre-invasive lesion, nodule or small mass, or having a solid malignant tumour, the method comprising:
   determining a ratio of activated and/or exhausted T cells:naïve and/or resting T cells in a sample of blood obtained from the subject,
   wherein the determining comprises analysing T cells using cytometry to detect the presence or absence of a panel of biomarkers comprising Ki67 and CD39, and
   wherein the subject is at risk if
   the ratio of activated and/or exhausted T cells:naïve and/or resting T cells is equal to or greater than
   a ratio of activated and/or exhausted T cells:naïve and/or resting T cells of a comparison subject, or
   an average ratio of activated and/or exhausted T cells:naïve and/or resting T cells of a plurality of comparison subjects,
   and
   providing treatment to said subject, if said subject is determined to be at risk.

The invention further provides a method of treating a subject determined to be at risk for having a progressing or high-grade pre-invasive lesion, nodule or small mass, or having a solid malignant tumour, wherein the method comprises:
determining whether a subject is at risk for having a progressing or high-grade pre-invasive lesion, nodule or small mass, or having a solid malignant tumour, the method comprising:
determining a ratio of activated and/or exhausted T cells:T cells which are not activated and/or exhausted T cells in a sample of blood obtained from the subject,
wherein the determining comprises analysing T cells using cytometry to detect the presence or absence of a panel of biomarkers comprising Ki67 and CD39, and
wherein the subject is at risk if
the ratio of activated and/or exhausted T cells:T cells which are not activated and/or exhausted is equal to or greater than a ratio of activated and/or exhausted T cells:T cells which are not activated and/or exhausted of a comparison subject, or
an average ratio of activated and/or exhausted T cells:T cells which are not activated and/or exhausted of a plurality of comparison subjects, and providing treatment to said subject, if said subject is determined to be at risk.

This invention further provides a method of treating a subject determined to be at risk for having a progressing or high-grade pre-invasive lesion, nodule or small mass, or having a solid malignant tumour, wherein the method comprises:
determining whether a subject is at risk for having a progressing or high-grade pre-invasive lesion, nodule or small mass, or having a solid malignant tumour, the method comprising:
   determining a ratio of activated and/or exhausted T cells:T cells which are not activated and/or exhausted T cells in a sample of blood obtained from the subject,
wherein the determining comprises analysing T cells using cytometry to detect the presence or absence of a panel of biomarkers comprising Ki67 and CD39, and
   wherein the subject is at risk if
   the ratio of activated and/or exhausted T cells:T cells which are not activated and/or exhausted is equal to or greater than
   a ratio of activated and/or exhausted T cells:T cells which are not activated and/or exhausted of a comparison subject, or
   an average ratio of activated and/or exhausted T cells:T cells which are not activated and/or exhausted of a plurality of comparison subjects,
   and
   providing treatment to said subject, if said subject is determined to be at risk.

The present invention also provides a method of treating a subject determined to be at risk for having a progressing or high-grade pre-invasive lesion, nodule or small mass, or having a solid malignant tumour, wherein the method comprises:
determining whether a subject is at risk for having a progressing or high-grade pre-invasive lesion, nodule or small mass, or having a solid malignant tumour, the method comprising:
determining the proportion of activated and/or exhausted T cells as a percentage of T cells in the sample of blood of a subject, wherein the determining comprises analysing T cells using cytometry to detect either (i) the presence of Ki67 and CD39 and/or (ii) the presence of FoxP3, wherein the activated and/or exhausted T cells are CD4 T cells,
and/or
wherein the subject is at risk if
the proportion of activated and/or exhausted T cells is higher than the proportion of activated and/or exhausted T cells in a comparison subject, or
the proportion of activated and/or exhausted T cells is higher than the average proportion of activated and/or exhausted T cells in a plurality of comparison subjects, and providing treatment to said subject, if said subject is determined to be at risk.

In some embodiments, the treating may comprise administering an anti-cancer therapeutic. In some embodiments, the treating may comprise administering a therapeutic suitable for treating pre-invasive neoplasia and/or a high grade pre-invasive lesion, nodule or small mass. In some embodiments, the treating may comprise administering a therapeutic suitable for treating a solid malignant tumour, for example, a stage I solid malignant tumour.

In some examples, the term “comprising analysing the proportion of T cells in a sample of blood obtained from the subject which are activated and/or exhausted T cells by analysing a trait of the T cells” may encompass or be used interchangeably with the term “determining a subject's T cell differentiation state in a blood sample”. Therefore, also disclosed herein is a method for determining whether a subject is at risk for having a progressing or high-grade pre-invasive lesion, nodule or small mass, or having a solid malignant tumour, the method comprising determining a subject's T cell differentiation state in a blood sample obtained from a subject. In some embodiments, a subject's T cell differentiation state is determined (i) using cytometry (optionally flow cytometry) to detect the presence or absence of a panel of biomarkers as described herein.

In some examples, the term “high-grade pre-invasive lesion, nodule or small mass” may encompass or be used interchangeably with the term “pre-invasive neoplasia”, “pre-invasive neoplastic lesion”, as well as other terms described elsewhere herein.

For any method defined herein describing a solid malignant tumour, the solid malignant tumour is a stage I solid malignant tumour (i.e., an early-stage solid malignant tumour). This is distinguished from more established tumours in later stages.

As used herein, the term “determining a proportion of activated and/or exhausted CD4 T cells as a percentage of CD4 T cells in a sample of blood obtained from the subject” may be used interchangeably with “determining a proportion of CD4 T regulatory cells as a percentage of CD4 T cells in a sample of blood obtained from the subject”.

### BRIEF DESCRIPTION OF THE DRAWINGS

For a better understanding of the present invention, and to show more clearly how it may be carried into effect, reference will now be made, by way of example, to the accompanying drawings, in which:

FIG. 1 shows the use of biomarkers (CD39, Ki67) to classify low from high grade pre-LUSC. The data uses a combination of CD4 and CD8 T cells. In this example the data is shown as a generated TEDI score that shows the capacity to classify low from high-grade pre-LUSC. A) Workflow for how to generate the TEDI score, B) result of non parametric Mann-Whitney, One-tailed test of the Minimal. TEDI score between low-grade and high-grade pre-LUSC patients as described above. C) Gating strategy for identifying cell populations required to generate the score and D) the associated calculation.

FIG. 2 shows a workflow showing the protocol of how the computational high dimensional analysis and clustering of the flow cytometry data was completed following the analysis steps outlined. The Figure shows each major step in how a skilled operator should analyse files from a flow cytometer using the R programming environment (https://www.r-project.org/) to obtain a list of all T cell clusters within a set of samples, and the proportion of each cluster in each sample, including the exhausted/activated and resting/naïve T cell clusters used in the invention. All packages described are freely available to download at https://cran.r-project.org/ or https://www.bioconductor.org/. The person skilled in the art will know that the workflow provided is merely an example in the context of the Examples provided herein and is not intended to limit the invention in any way.

FIG. 3 shows that systemic T cell differentiation skewing distinguishes patients with high vs low-grade pre-invasive central airway lesions. A) Flow cytometry data from PBMCs showing Uniform Manifold Approximation Projections (UMAPs) of CD4 (left) and CD8 (right) viable T cells from 66 samples (31 high vs 35 low grade) of 30 patients (14 high vs 16 low Grade) with pre-invasive lesions. This shows which clusters of T cells are present across all samples and their relative abundance. Individual clusters and major subsets of cells are indicated. B) Heatmaps of biomarker expression in each cluster for CD4 and CD8 T cells. This shows that there are 31 different clusters T cell present in the blood of patients with pre-LUSC, and details which biomarkers describe those different clusters. Clusters are in rows and biomarkers are in columns. For each cluster the drawing shows whether a biomarker is highly, lowly, or intermediately expressed. The lines on the left are a dendrogram, which shows how similar clusters of T cells are related. All clusters within CD8 T cells are on the left heatmap, and those within CD4 T cells are on the right. This shows that there are multiple types of activated/exhausted and resting/naïve T cell present in the pool of samples. C) Volcano plots showing significant differences in cluster frequency for CD8 (left) and CD4 (right) T cells. Clusters enriched in high-grade disease are shown on the left of each plot and those enriched in low grade disease on the right. Samples from patients that progressed or regressed between lesion grades were excluded for analysis. This shows which clusters of T cell are increased in high-grade disease, and which are increased in low grade disease. Activated/exhausted T cells are increased in high-grade and resting/naïve are increased in low grade disease. D) The ratio of clusters enriched in high vs low grade (activated/exhausted vs resting progenitor) was developed as a potential classifier and plotted per patient for CD8 (left), CD4 (centre) or all T cell subsets (right). This shows that the ratio of activated/exhausted:naïve/resting T cells is significantly higher in high vs low grade disease. E) Receiver operator characteristic curves (ROCs) for the ratio of activated/exhausted:naïve/resting T cells within CD4 and CD8 T cells, labelled as CD4 TEDI and CD8 TEDI, respectively. This shows that 92-94% of patients can be correctly classified as High or Low-grade using the invention. P values from one-tailed, unpaired Wilcoxon test. E) ROC curves of the metrics from flow cytometry and data indicated, AUC, Area Under Curve. TEDI, T cell early detection index.

FIG. 4 shows CD4 T cell differentiation skewing in pre-invasive lung neoplasia. A) Summary of biomarker expression in clusters that show significantly different abundance in high vs low grade disease according to sample level analysis. The mean frequency of each cluster was calculated per patient and shown in B) volcano plot and c) bar plots. D) Cluster names, median biomarker expression and annotation of enrichment in high-grade or low-grade. This Figure shows which clusters of T cell are present amongst CD4 T cells and highlights those that are significantly different in high or low-grade disease in detail, indicating expression of key biomarkers CD39 and Ki67 and additional biomarkers PD1, CCR7, CD45RA, CD57 and CD38.

FIG. 5 shows CD8 T cell differentiation skewing in pre-invasive lung neoplasia. A) Summary of biomarker expression in clusters that show significantly different abundance in high vs low grade disease according to sample level analysis. The mean frequency of each cluster was calculated per patient and shown in B) volcano plot and c) bar plots. D) Cluster names, median biomarker expression and annotation of enrichment in high-grade or low-grade. This Figure shows which clusters of T cell are present amongst CD8 T cells and highlights those that are significantly different in high or low-grade disease in detail, indicating expression of key biomarkers CD39 and Ki67 and additional biomarkers PD1, CCR7, CD45RA, CD57 and CD38.

FIG. 6 shows a basic workflow showing how to take a blood sample and measure the change in CD4 or CD8 clusters as a single score referred to as the T cell early detection index (TEDI). To generate TEDI, a ratio of [sum freq. of all CD4 T cell clusters enriched in HG disease]/[sum freq. of all clusters enriched in LG disease] was calculated and the process was repeated for CD8 T cells. Activated/exhausted T cells were increased in high-grade and resting/naïve are increased in low-grade disease. This is a key step in using the invention. The person skilled in the art will know that the workflow provided is merely an example in the context of the Examples provided herein and is not intended to limit the invention in any way.

FIG. 7 shows T cell early detection indices (TEDI) in high vs low-grade disease. Clusters associated with high-grade disease were summed and divided by the sum frequency of clusters associated with low grade disease to generate a single score referred to as the TEDI. This was calculated per patient according to clusters in CD4 or CD8 T cells or the average of both (combined). Each dot is a patient, p values from one way Wilcoxon tests, dotted lines represent the sensitivity adjusted cut-off values in the table below. This drawing summarises the key results from the example, showing that the ratio of exhausted/activated:naïve resting T cells is significantly higher in high vs low-grade disease. The person skilled in the art will know that the use of a TEDI provided is an in-house term that is specific to the inventors and that various methods and techniques discussed herein can be performed using a variety of different apparatus, assay conditions, computational hardware and software components that may result in different numerical values obtained, but which equate to the same biological results to the Examples provided herein. Therefore, the use of a TEDI is merely an example in the context of the Examples provided herein and is not intended to limit the invention in any way.

FIG. 8 shows the gating strategy for manual identification of high and low grade associated T cell populations. Gating (from left to right upper panel) shows identification of live, single, CD3+ lymphocytes, within live T cells CD4 and CD8 T cells were identified and then gating of key CD8 and CD4 T cell subsets that were enriched in high-grade samples and low-grade samples. This drawing shows how a skilled researcher could bypass computational analysis to identify several activated/exhausted and naïve/resting T cell clusters manually using compensated FCS (Flow Cytometry Standard) files in FlowJo software.

FIG. 9 shows data from RCC patients showing an increase in systemic T cell differentiation in malignant (n=16 samples from 10 patients) vs benign (n=3 samples from 3 patients) disease. A) UMAP of FlowSOM defined T cell clusters from PBMC of all samples stained with 31 biomarkers and analysed by spectral cytometry. 5000 live CD3+ events per samples were down-sampled for analysis. B) The ratio of progenitor vs exhausted CD4 (left), CD8 (centre) or combined (right) T cell subsets. P values from one-tailed, unpaired Wilcoxon test. This drawing shows that the ratio of activated/exhausted:naïve/resting T cells is increased in the blood of patients with renal cancer vs patients with benign disease.

FIG. 10 Top) depicts the pre-invasive data and shows the T cell early detection indices (TEDI) in high grade pre-invasive samples versus low grade samples, Bottom) shows the T cell early detection indices (TEDI) in healthy patients versus NSCLC (LUAD+LUSC) patients (the majority of which have stage I disease) deriving from the ASCENT analysis. Each datapoint is a patient and the p-value is from a one-tailed MW test. This is demonstrated by the TEDI CD39 Ki67 (CD3), where NSCLC patients have a higher proportion of CD3 CD39+Ki67+ cells than healthy.

FIG. 11 shows the T cell early detection indices (TEDI) of the pre-invasive data (i.e., high grade and low-grade samples) and the ASCENT data (i.e., healthy and NSCLC samples) combined. Each datapoint is a patient with the median value shown (top) or the mean of all patients in each group (bottom) and p-values are from a Kruskal-Wallis test, where only significant values are shown. Error bars represent SEM. An increase in the TEDI CD39 Ki67 ratio from healthy samples to low-grade samples to high-grade samples is observed, with the signal peaking at high-grade samples and dropping off at the lung cancer stage.

FIG. 12 shows the lower frequency of naïve CD4+ T cells in NSCLC patients compared with healthy patients (p=0.1, one-tailed MW test). Naïve CD4+ T cells are shown as a percentage of effector CD4+ T cells (eCD4), meaning non-regulatory CD4+ T cells, or FoxP3− CD4+ T cells.

FIG. 13 shows the combined data for (i) healthy and low grade samples (n=66) compared with (ii) high-grade pre-invasive samples and NSCLC patients (N=88). T cell early detection indices (TEDI); % Total Treg, % Treg CD39+, % Tref CD39+Ki67+ and % naïve CD4+ T cells as a proportion of total CD4 cells are all provided. p-values are all from a one-tailed MW test.

FIG. 14 shows the % Treg, Treg CD39+ and Treg CD39+Ki67+ as a proportion of total CD4 cells for both high grade pre-invasive and low grade samples (top) and for healthy and NSCLC patients (bottom). Each datapoint is a sample (top) or patient (bottom) and the p-value is derived from a one-tailed MW. Treg stands for a T regulatory cell, which are CD4+ T cells that express the biomarker FoxP3.

FIG. 15 shows the % Treg, Treg CD39+ and Treg CD39+Ki67+ as a proportion of total CD4 cells for healthy, low grade pre-invasive samples, high grade pre-invasive samples and NSCLC patients. Each datapoint is a patient (healthy/NSCLC) or sample (low-/high-grade) and uncorrected KW p-values are shown for all. Treg stands for a T regulatory cell, which are CD4+ T cells that express the biomarker FoxP3.

FIG. 16 shows a volcano plot of CD8+ T cell populations determined by computational clustering of flow cytometry data of pre-invasive samples that are significantly enriched in high grade (HG; upper right quadrant) pre-invasive or low grade (LG, upper left quadrant) samples.

FIG. 17 shows box plots for naïve and Tem.Prolif.CF39hi populations at the sample level for both patients with both low grade and high grade pre-invasive species. Tem.Prolif.CF39hi cells are significantly enriched in high grade pre-invasive samples, and naïve cells are significantly enriched in low grade pre-invasive samples. Both are shown as a proportion of total CD8+ T cells.

FIG. 18 Top) shows a volcano plot of CD8+ T cell populations determined by computational clustering of flow cytometry data that are significantly enriched in LUSC (upper right quadrant). Bottom) shows a box plot for the Tem.Prolif.CF39hi population which is significantly enriched in LUSC patients compared to a healthy group.

FIG. 19 shows a combined box plot of values from the pre-invasive flow cytometry analysis and the ASCENT flow cytometry analysis to display the change in Tem. Prolif. CD39hi population. Each dot represents a patient (healthy/LUSC) or sample (low/high). Shown as a proportion of total CD8+ T cells.

FIG. 20 shows the flow cytometry gating strategy of CD8+ T cells for the pre-invasive data samples and ASCENT data samples used in Example 6 and FIGS. 17-19. Manual gating frequencies are used to validate results from computational clustering.

FIG. 21 shows the flow cytometry panel used for analysis of the ASCENT data-set.

FIG. 22 shows another example flow cytometry manual gating strategy for the manual gating analysis of the pre-invasive CD4+ T cell data (i.e., high grade and low grade pre-invasive samples) and ASCENT flow cytometry CD4+ T cell data (i.e., healthy subjects and NSCLC patients), used in Example 5, including analysis that selects for regulatory T cells using the biomarker FoxP3. All manual gating analysis was carried out on FlowJo v10.8.1. Populations gated are shown on a concatenated file of all samples from one batch. Frequencies from total CD4 were used to validate cluster significance from the high-dimensional clustering pipeline analysis.

FIG. 23 Top) shows the % Treg CD45RA-CD39+ as a proportion of total CD4 cells for both high and low grade pre-invasive samples (left) and for healthy and NSCLC patients (right). Bottom shows a combined data from the pre-invasive flow cytometry analysis and the ASCENT flow cytometry to display the change in Treg CD45RA-CD39+ population.

FIG. 24 shows a Forest plot displaying odds ratios calculated from multivariate analysis logistic regression models accounting for listed clinical variables in the pre-invasive data. This analysis shows that metrics from flow cytometry (Treg CD45RA-CD39+) remain significant even after accounting for the listed clinical variables.

### DETAILED DESCRIPTION

### Summary of how the Problem is Solved

The present invention provides a blood test which examines the state of blood T cell differentiation to determine whether a subject has or is at risk for having a progressive or high-grade pre-invasive lesion or nodule or small mass. It is understood that progressive or high-grade pre-invasive lesions or nodules or small masses are of concern because they can develop into a solid malignant tumour. The methods of the present invention can also provide a blood test which examines the state of blood T cell differentiation to determine whether a subject has or is at risk of having a solid malignant tumour. This is because similar or the same patterns of T cell differentiation skewing may be identified if a subject has a solid malignant tumour. Therefore, the present invention provides a method for determining whether a subject is at risk for having a progressing or high-grade pre-invasive lesion, nodule or small mass, and/or having a solid malignant tumour, by analysing T cell differentiation skewing in a sample of blood obtained from the subject. Prior to the present invention there was no effective non-invasive screening method for early detection of a progressive or high-grade pre-invasive lesion or nodule or small mass that uses T cell differentiation for the test.

T cells in humans with a challenged immune system are known to look more activated, exhausted or differentiated, compared to those who are not challenged. Prior to the present invention it was not known that such a challenged immune system could be detected in individuals who do not yet have an established solid malignant tumour but harbour a lesion or nodule or small mass of the type that can be expected to develop into disease. The present invention leverages this concept by generating a test that measures T cell differentiation, exhaustion and activation.

The present invention is also concerned with early detection of cancer. Other “early detection” methods have been concerned with detection of cancer at stage 1 or 2, rather than detection at stage 3 or 4 when the prognosis is very poor, and this has sometimes been referred to as “early detection”. In contrast early detection in the context of the present invention includes detection of pre-invasive or high-grade lesions or nodules that can develop into a solid tumour. Detection prior to the establishment of a tumour can lead to greater treatment options and improved prognosis.

The present invention may be used for detection of a progressing or high-grade pre-invasive lesion or nodule. The present invention may also be valuable to detect solid malignant tumours at stage I or later.

Pre-invasive generally refers to a cluster of malignant cells or lesions that have not left their original focal or spread to other parts of the body and are not yet considered to be invasive. Nodule generally refers to a growth or lump that may be malignant or benign. The early cancer detection in the present disclosure is concerned with changes prior to stages 1-4 of established cancer. Therefore, pre-invasive as described herein may refer to a stage of neoplasia development before stages 1-4 cancer. Optionally, this disclosure may be concerned with very early (stage 1) solid tumours, i.e., the detection of very early (stage 1) solid tumours.

In the context of this invention, pre-invasive lesions or nodules or small masses are or can be classified into low-grade and high-grade.

High-grade pre-invasive lesions, nodules or small masses have a well-established meaning in the art. This is demonstrated in at least by Pennycuick et al; Cancer Discov. 2020 October; 10(10): 1489-1499; Banerjee et al; Journal of Thoracic Oncology, Volume 4, Issue 4, April 2009, Pages 545-551; D. Moro-Sibilot et al; European Respiratory Journal 2004 24: 24-29, among others.

The term high-grade pre-invasive lesion, nodule or small mass may also encompass or may be used interchangeably with pre-invasive neoplasia and pre-invasive neoplastic lesion. In some embodiments, the term high-grade pre-invasive lesion, nodule or small mass may also encompass or be used interchangeably with the following terms in the Table below:

High grade pre-malignant lesions
High grade preinvasive neoplasia
Severe dysplasia
Carcinoma-in-situ (CIS)
Early lesion, nodule or small mass
Pre-malignant lesion, nodule or small mass
Pre-cursor lesion, nodule or small mass
High-grade dysplasia
Squamous dysplasia
Minimally invasive lesion, nodule or small mass
Stage 0 lesion, nodule, small mass, or tumour
Progressive preinvasive disease
Progressive preinvasive neoplasia
Any of the above with tissue type specific
prefixes, suffixes or combinations which
indicate disease involved in carcinogenesis
for different cancer types (e.g. pulmonary
or bronchial preinvasive neoplasia when
referring to disease that results in lung cancer).

High-grade lesion is an umbrella term encompassing carcinoma in situ (CIS) and severe dysplasia lesions of the airway. Low-grade lesions include hyperplasia, squamous metaplasia, mild dysplasia, and moderate dysplasia. These are all different histological states of different levels of neoplasia of the airway that can be identified from a lesion biopsy by a histopathologist. These different histological states are classified into low- or high-grade groups to facilitate downstream analysis, and separated in this way based on the clinical risk each group carries. Low-grade lesions carry no significant clinical risk of developing into invasive disease/lung cancer, whereas 50% of high-grade lesions have been reported to progress into invasive disease/lung cancer (P J George et al. Surveillance for the detection of early lung cancer in patients with bronchial dysplasia. Thorax 2007; 62:43-50. doi: 10.1136/thx.2005.052191).

In some embodiments, the high grade pre-invasive lesion, nodule or solid mass may be a high-grade bronchial lesion, a pre-invasive lesion of the bronchus, a bronchial pre-invasive lesion, a bronchial dysplasia, bronchial lesion or an endobronchial lesion.

In the present invention, classification of lesions or nodules or small masses into low-grade or high-grade can be performed using a ratio of differing T cell phenotypes, identified using detection biomarkers on/within the T cells. Classification of lesions or nodules or small masses into low-grade or high-grade can be performed by determining the proportion of T cells comprising one or more detection biomarkers on/within the T cells as a percentage of T cells.

Skewing of T cell differentiation can be detected by determining a ratio of activated and/or exhausted T cells:naïve and/or resting T cells in a sample of blood obtained from the subject, or by determining a proportion of activated and/or exhausted T cells as a percentage of T cells (i.e., total T cells) or a subpopulation of T cells (i.e., total subpopulation of T cells). Optionally skewing of T cell differentiation can be detected by determining a ratio of activated and/or exhausted T cells:T cells which are not activated and/or exhausted T cells in a sample of blood obtained from the subject. Where a subject is shown to have more activated and/or exhausted T cells based on detection of a combination of T cell biomarker expression, this would be indicative of the subject being at risk of having a progressing or high-grade pre-invasive lesion or nodule or small mass, or having a solid malignant tumour (e.g., a stage 1 solid malignant tumour). Optionally skewing of T cell differentiation can be detected by determining a proportion of activated and/or exhausted T cells as a proportion of T cells, or a subpopulation of T cells, in a sample of blood obtained from the subject. Where a subject is shown to have a greater proportion of activated and/or exhausted T cells (e.g., based on detection of a specific biomarker expression), this would be indicative of the subject being at risk of having a progressing or high-grade pre-invasive lesion or nodule or small mass, or having a solid malignant tumour (e.g., a stage 1 solid malignant tumour).

In the context of the invention, Low-grade or “LG”, as used herein, generally refers to the pre-invasive stages of cancer development characterised by squamous metaplasia, mild dysplasia and moderate dysplasia. Generally, a low-grade pre-invasive lesion or nodule or small mass may not need further clinical intervention since it is not expected to develop into cancer. This has a well-established term in the art.

Whereas high-grade or “HG”, as used herein, generally refers to the pre-invasive disease stages of cancer development characterised by severe dysplasia and carcinoma in situ. Generally, a high-grade pre-invasive lesion or nodule or small mass should be the focus of further clinical follow up. This has a well-established term in the art. This term may encompass or may be used interchangeably with pre-invasive neoplasia and/or other terms as described elsewhere herein.

Provided in the present disclosure is an example of generating a ratio of T cell differentiation that can be used to classify a subject as having non-progressing or low-grade pre-invasive lung lesions (posing no risk of cancer) or a subject having a progressing or high-grade pre-invasive lung lesions (high risk of developing cancer). Also provided in the present disclosure is an example of measuring the extent of T cell differentiation by determining a proportion of T cells that comprise or do not comprise a panel of biomarkers, as a percentage of T cells. This can also be used to classify a subject as having non-progressing or low-grade pre-invasive lung lesions (posing no risk of cancer) or a subject having a progressing or high-grade pre-invasive lung lesions (high risk of developing cancer).

The utility of the present invention is shown herein in two types of lung cancer with differing clinical and genomic features and in renal cancer. These are cancers with differing body locations and differing mutational backgrounds. Therefore, the underlying principle of T cell activation or T cell differentiation skewing in the presence of a high-grade or progressing pre-invasive nodule, or lesion, which could lead to a malignant tumour is broad and encompasses solid tumours generally. Therefore the utility of this test for early detection is pan-cancer.

### Advantages

The present invention provides at least the following advantages over previously known methods.
A non-invasive method for detecting whether a subject is at risk for having a progressing or high-grade pre-invasive lesion or nodule or having a solid tumour using T cells as a read out.
A method for distinguishing between a progressing or high-grade pre-invasive lesion or nodule and a non-progressing or low-grade lesion or nodule.
An in vitro method.
A method that only requires a blood sample to determine whether a subject is at risk. Whole blood or peripheral blood mononuclear cell (PBMC) may be used.
A non-invasive procedure that encourages high compliance with a population screening program requiring.
A method that has high sensitivity and specificity.
A method that is valuable for early detection of a wide range of solid tumours, such as RCC, LUAD and LUSC which therefore provides a valuable tool as the test works on a variety of cancers, even when they come from different causes.
A method that can be used as a screening tool on healthy people that can be used to see if the subject is healthy and can be incorporated in standard blood tests to see the likelihood that someone has a risk of progressing or high-grade pre-invasive lesion or nodule or having a solid tumour.
A method that can be personalised to be used as subject's baseline, that can be referred to in later tests to see if the subject's risk of developing progressing or high-grade pre-invasive lesion or nodule or having a solid tumour

Unless defined otherwise, all technical and scientific terms used herein have the meaning commonly understood by a person skilled in the art to which this invention belongs. Certain terms are defined below for the sake of clarity and ease of reference.

The present invention provides a method or methods for determining whether a subject is at risk for having a progressing or high-grade pre-invasive lesion or nodule or having a solid tumour. This or these methods can also be understood as distinguishing a subject having a progressing or high-grade pre-invasive lesion or nodule or having a solid tumour, from a subject not having a progressing or high-grade pre-invasive lesion or nodule or not having a solid tumour or having a non-progressing or low-grade pre-invasive lesion or nodule. The method or methods disclosed herein also can distinguish a subject having a progressing or high grade pre-invasive lesion or nodule, or having a solid tumour (e.g., a stage I solid tumour) from a healthy subject.

The subject is a mammal. The subject may be a human. Alternatively the subject may be in the Primates, Rodentia, Canidae, Felidae, Equidae order or other mammal orders. The subject may be a horse, cat, dog or other companion animal. The subject can be healthy or asymptomatic. The subject may be a patient. The subject can be suspected of having a cancer. The subject can have a genetic pre-disposition to cancer or have lifestyle factors increasing the likelihood for developing cancer. The subject may have previously received treatment for a cancer. The methods of the present invention may also be used for population screening and/or be used as a standard clinical tool in routine blood work, also referred to as mass testing.

A subject is at risk, if the method of the present invention indicates that the subject is likely to have, or has, a high-grade pre-invasive lesion or nodule. A subject is also at risk if the method of the present invention indicates that the subject is likely to have, or has, a solid tumour, e.g., a stage 1 solid malignant tumour.

The term solid tumour generally refers to an abnormal mass of tissue that usually does not contain cysts or liquid areas. Solid tumours may be benign, or malignant. The methods of the present allow to determine if a subject is at risk of developing a solid malignant tumour or at risk of having a solid malignant tumour (e.g., a stage I solid tumour). The term is meant to exclude liquid tumours or haematological malignancies. A lesion generally refers to an area of abnormal tissue. A lesion may be benign or malignant, or premalignant and if premalignant can represent moderate or mild or severe dysplasia, metaplasia or carcinoma in situ. A nodule generally refers to a growth or lump that may be malignant, benign or indeterminate. Low-grade or “LG”, as used herein, generally refers to the pre-invasive disease stages of cancer development characterised by squamous metaplasia, mild dysplasia and moderate dysplasia. Whereas high-grade or “HG”, as used herein, generally refers the pre-invasive disease stages of cancer development characterised by to severe dysplasia and carcinoma in situ. This has a well-established term in the art. This term may encompass or may be used interchangeably with pre-invasive neoplasia and/or other terms as described elsewhere herein.

The methods of the present invention are performed on a blood sample obtained from a subject and hence occur in vitro. The methods can use whole blood. The methods can use PBMCs.

In an embodiment of the first aspect of the invention, or in a second aspect of the invention, the method of the present invention comprises determining a ratio of activated and/or exhausted T cells:naïve and/or resting T cells in a sample of blood obtained from the subject, wherein the determining comprises analysing T cells using cytometry to detect the presence or absence of a panel of biomarkers comprising Ki67 and CD39.

In an embodiment of the first aspect of the invention, or optionally in the method of the second aspect of the present invention, or in a third aspect of the invention, the method comprises determining a ratio of activated and/or exhausted T cells:T cells which are not activated and/or exhausted T cells in a sample of blood obtained from the subject, wherein the determining comprises analysing T cells using cytometry to detect the presence or absence of a panel of biomarkers comprising Ki67 and CD39.

A subject having more T cells showing Ki67 and CD39 expression, vs T cells in which there is no detection of expression of one or both of Ki67 and CD39, in comparison with a comparison subject or a plurality of comparison subjects is considered to be at risk of having a progressing or high-grade pre-invasive lesion or nodule. It is also noted that a subject having more T cells showing Ki67 and CD39 expression, vs T cells in which there is no detection of expression of one or both of Ki67 and CD39, in comparison with a comparison subject or subjects is considered to be at risk of having a solid tumour. In this context the comparison subject or subjects can include:
a healthy subject or a plurality of healthy subjects,
the subject at a different time point,
a plurality of subjects from the general population.

A subject having the same amount or more T cells showing Ki67 and CD39 expression, vs T cells in which there is no detection of expression of one or both of Ki67 and CD39, in comparison with a comparison subject or a plurality of comparison subjects is considered to be at risk of having a progressing or high-grade pre-invasive lesion or nodule. It is also noted that a subject having the same amount or more T cells showing Ki67 and CD39 expression, vs T cells in which there is no detection of expression of one or both of Ki67 and CD39, in comparison with a comparison subject or subjects is considered to be at risk of having a solid tumour. In this context, the comparison subject or subjects can include:
a subject or a plurality of subjects known or have a solid tumour or known to have a progression or high-grade pre-invasive lesion or nodule,
the subject at a different time point,
a plurality of subjects from the general population.

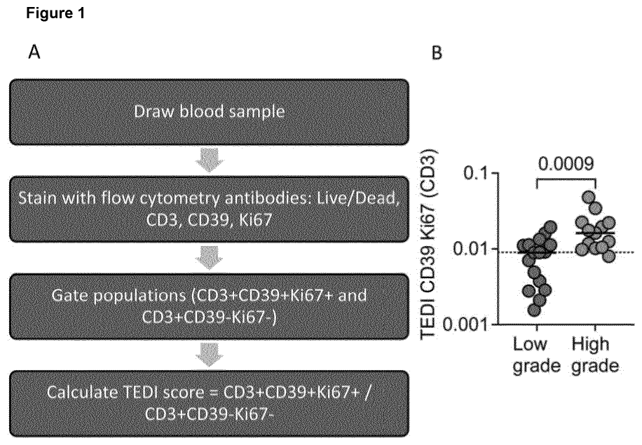

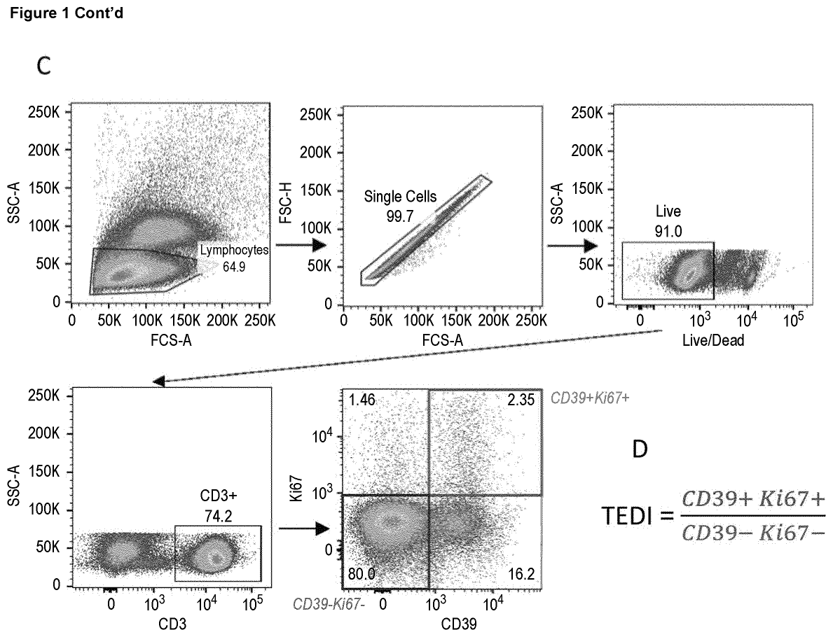

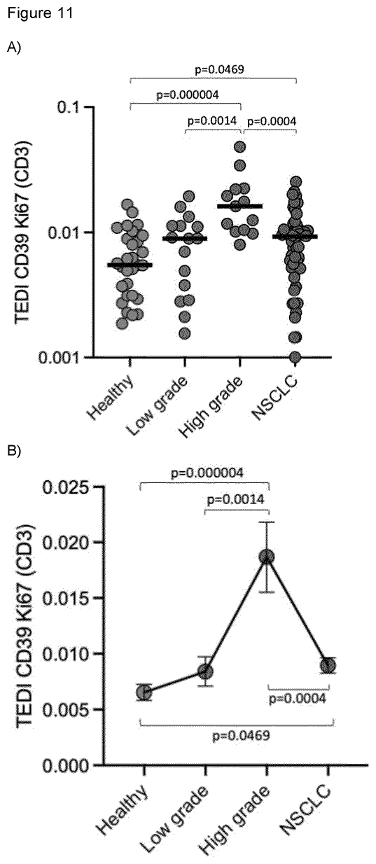

The validity of comparing results in this way is supported through the data provided herein where subjects with known progressing or high-grade pre-invasive lesion or nodule or having a solid tumour have more T cells showing Ki67 and CD39 expression, and conversely, subjects with low-grade disease have less T cells with Ki67 and CD39 expression (FIG. 1) as well as healthy subjects (FIG. 11).

In an embodiment of the first aspect of the invention, or in the method of the fourth aspect of the present invention, or in method of the fifth aspect of the invention, the comprises determining a proportion of activated and/or exhausted T cells as a percentage of T cells.

In an embodiment of the first aspect of the invention, or in the method of the fifth aspect of the invention, the method of the present invention comprises determining the proportion of activated and/or exhausted T cells as a percentage of T cells in the blood obtained from the subject, wherein the determining comprises analysing T cells using cytometry to detect the presence or absence of a panel of biomarkers comprising FoxP3, wherein the T cells are CD4 T cells. In some embodiments, the panel of biomarkers comprises CD4 to identify the CD4 T cells also using cytometry. In some embodiments, the CD4 T cells are pre-selected for before the cytometry step, e.g., using a T cell enrichment or purification kit and magnetic selection (as described elsewhere herein). In some embodiments, the panel of biomarkers further comprises CD39. In some embodiments, the panel of biomarkers further comprises CD39 and Ki67, or CD39 and CD45RA. In some embodiments, the activated and/or exhausted T cells express FoxP3 and CD39, wherein the T cells are CD4 T cells. In some embodiments, the activated and/or exhausted T cells express FoxP3 and CD39 in combination with Ki67, wherein the T cells are CD4 T cells. In some embodiments, the activated and/or exhausted T cells express FoxP3 and CD39 and do not express CD45RA, wherein the T cells are CD4 T cells.

In another embodiment, the method of the present invention comprises determining the proportion of regulatory T cells as a percentage of T cells in the blood obtained from the subject, wherein the determining comprises analysing T cells using cytometry to detect the presence or absence of a panel of biomarkers comprising FoxP3, wherein the T cells are CD4 T cells. In some embodiments, the activated and/or exhausted T cells express FoxP3 and CD39 in combination with Ki67, wherein the T cells are CD4 T cells. In some embodiments, the activated and/or exhausted T cells express FoxP3 and CD39 and do not express CD45RA, wherein the T cells are CD4 T cells.

A subject having more CD4 T cells showing FoxP3 expression (preferably in combination with CD39 expression and further preferably in combination with (i) Ki67 expression, or (ii) no detection and/or no expression of CD45RA), vs CD4 T cells in which there is no detection of expression of FoxP3 (preferably in combination with no detection or expression of CD39, and further preferably in combination with (i) no detection or expression of Ki67, or (ii) expression and/or detection of CD45RA, in comparison with a comparison subject or a plurality of comparison subjects is considered to be at risk of having a progressing or high-grade pre-invasive lesion or nodule. It is also noted that a subject having more CD4+ T cells showing FoxP3 expression (preferably in combination with CD39 expression and further preferably in combination with (i) Ki67 expression, or (ii) no detection and/or expression of CD45RA), vs CD4+ T cells in which there is no detection of expression of FoxP3 (preferably in combination with no detection or expression of CD39, and further preferably in combination with (i) no detection or expression of Ki67, or (ii) expression of CD45RA), in comparison with a comparison subject or subjects is considered to be at risk of having a solid tumour (e.g., a stage 1 solid tumour). In this context the comparison subject or subjects can include:
a healthy subject or a plurality of healthy subjects,
the subject at a different time point,
a plurality of subjects from the general population.

In an embodiment of the first aspect of the invention, or in the method of the fourth aspect of the invention, the method of the present invention comprises determining the proportion of activated and/or exhausted T cells as a percentage of T cells in the blood obtained from the subject, wherein the determining comprises analysing T cells using cytometry to detect the presence or absence of a panel of biomarkers comprising CD39 and Ki67. A subject having the same amount or more T cells showing Ki67 and CD39 expression, vs T cells in which there is no detection of expression of one or both of Ki67 and CD39, in comparison with a comparison subject or a plurality of comparison subjects is considered to be at risk of having a progressing or high-grade pre-invasive lesion or nodule. It is also noted that a subject having the same amount or more T cells showing Ki67 and CD39 expression, vs T cells in which there is no detection of expression of one or both of Ki67 and CD39, in comparison with a comparison subject or subjects is considered to be at risk of having a solid tumour. In this context, the comparison subject or subjects can include:
a subject or a plurality of subjects known or have a solid tumour or known to have a progression or high-grade pre-invasive lesion or nodule,
the subject at a different time point,
a plurality of subjects from the general population.

The methods of the present invention are valuable for early detection of a variety of solid tumours. Early detection may be for NSCLC including LUAD or LUSC, mesothelioma, renal cancer, melanoma, pancreatic cancer, head and neck cancer, prostate cancer, brain cancer including glioblastoma, breast cancer, bowel cancer, liver cancer.

A ratio as used herein, generally refers to the relationship of the frequency of activated and/or exhausted T cells compared with the frequency of naïve and/or resting T cells in a subject. Alternatively, a ratio can refer to the relationship of the frequency of activated and/or exhausted T cells amongst all T cells or amongst a subset of T cells in a subject (i.e., the proportion of activated/exhausted cells as a percentage of T cells, or amongst a subset of T cells in a subject, e.g., in a sample of blood in a subject).

A ratio indicative of a whether a subject is at risk can be inferred from having a high percentage of activated/exhausted T cells in a sample obtained from the subject.

A percentage of activated/exhausted T cells in a sample obtained from the subject which is greater than the average percentage of activated and/or exhausted T cells in a comparison subject or plurality of subjects is indicative of a subject at risk.

An activated T cell as used herein, generally refers to a T cell that has recognised its cognate antigen, on a target cell expressing the antigen including an epithelial cell or other cell that may progress to cancer or be part of a lesion or nodule or tumour triggering activation and/or proliferation of that T cell. Repetitive encounter of an antigen or a high affinity interaction with that antigen can cause terminal differentiation. Chronic antigen stimulation with or without inhibitory signalling or defective co-stimulation with or without inflammation may result in T cell exhaustion or T cell dysfunction. An activated T cell may or may not be proliferating.

Activated T cells may include:
Any subset of CD4 or CD8 T cell or CD3 positive cell expressing CD39 and/or Ki67 in the presence or absence of other commonly used activation markers obvious to a skilled researcher.
CD8 Terminally differentiated effector memory cells (RA) that are activated (TEMRA.Act). Such cells may have one or more of the biomarkers CD39, Ki67 CD45RA or CD57.
CD4 Regulatory T cells that are proliferating and may express PD-1 (Teg.prolif.PD-1) and may express one or more of the biomarkers CD39, Ki67 or PD-1, (along with FoxP3).
CD4 Effector memory CD4 T cells (TEM) which may express one or more of the biomarkers CD39 and Ki67. Such cells may lack the biomarkers CCR7 and CD45RA.
CD4 Regulatory T cells (Treg) which may express one or more of the biomarkers CD39 and Ki67, (along with FoxP3).
CD4 Proliferating regulatory T cells (Treg.prolif) may express one or more of the biomarkers CD39, Ki67hi or PD-1.
CD4 Proliferating effector memory CD4 T cells (Tem.Prolif) which may express one or more of the biomarkers CD39, Ki67hi and such cells may lack one or more of the biomarkers CCR7 and CD45RA.
cytolytic CD4 T cells (Cytolytic) which may be terminally differentiated and cytotoxic CD4 T cells and may have the biomarker CD57.

In some embodiments, an activated and/or exhausted T cell expresses one or more of the biomarkers Ki67, CD39 and FoxP3. An activated and/or exhausted T cell may express CD39 and Ki67 (CD39+ and Ki67+ T cells). Detection of expression of CD39 and Ki67 is considered to indicate a T cell that is activated and/or exhausted. An activated and/or exhausted T cell may also be a CD4 Regulatory T cell. A CD4 Regulatory T cell expresses the biomarker FoxP3. In some embodiments, the CD4 Regulatory T cell also expresses CD39 (i.e., in combination with FoxP3). Detection of FoxP3 expression (and preferably in combination with CD39) can indicate a CD4 T cell that is activated and/or exhausted. A CD4 T cell that expresses FoxP3 is otherwise referred to as a T regulatory cell herein. In some embodiments, the CD4 Regulatory T cell may express FoxP3, Ki67 and CD39. In some embodiments, the CD4 Regulatory T cell may express FoxP3, CD39, and may lack CD45RA.

A proliferating T cell, as used herein, generally refers to a T cell that has interacted with their cognate antigen or other activating stimuli (thereby becoming activated) and has increased, are increasing or in the process of increasing the numbers of that T cell through cell growth and division. A proliferating T cell is usually activated but may not always be.

An exhausted T cell as used herein, generally refers to a state of T cell dysfunction which can be defined by biological changes in the T cell, including poor effector function and/or expression of inhibitory receptors and a transcriptional state and/or phenotype and/or epigenetic state which is distinct from that of effector or memory or naïve T cells. Exhausted T cells may be referred to as Tex cells, dysfunctional T cells or Tdys cells.

Exhausted T cells may include:
exhausted, proliferating CD8 T cells (Tex.Prolif) which may express one or more of the biomarkers PD-1, Ki67 or CD39.
exhausted, proliferating CD4 T cells (Tex.Prolif) which may express one or more of the biomarkers PD-1, Ki67hi or CD39.
progenitor, exhausted CD4 T cells (TPEX), an early version of exhausted CD4 T cells and may express one or more of the biomarkers CD39, Ki67 or PD-1.

A naïve T cell, as used herein, generally refers to a T cell, which may be a CD4+ helper T cell or a CD8+ cytotoxic T cell, that has not yet encountered their cognate antigen.

Naïve T cells may include:
naïve CD8 T cells (Naïve) which may express one or more of the biomarkers CD45RA, CCR7.
naïve CD4 T cells that may have lost TCF7 expression (Naïve.TCF7neg), and/or which may express one or more of the biomarkers CD45RA, CCR7 or CD27.

CD4 native T cells (Naïve) which may express one or more of the biomarkers CD45RA and CCR7.

A resting T cell, as used herein, generally refers to a quiescent T cell that has stopped or is not proliferating or expressing biomarkers of activation.

Resting T cells includes:
CD8 terminally differentiated effector memory cells (RA) (TEMRA.Rest) which may express the biomarker CD45RA. Such cells may lack one or more of the biomarkers CCR7 and CD57.
resting central memory CD4 T cells which are an early differentiated resting memory cells that are unstimulated, which may express the biomarker CCR7. Such cells may lack one or more of CD45RA and CD38.

The methods of the present invention can detect biomarkers using cytometry, which as used herein, generally refers to techniques for measurement of properties of the cells. Cytometry embraces flow cytometry, spectral cytometry and mass cytometry (including time of flight cytometry or cytometry by time of flight (CyTOF).

The cytometry can be flow cytometry. Flow cytometry refers to a well-established technique that can be used to detect and analyse populations of cells and provide information regarding the physical or chemical characteristics of those cells. This detection and/or analysis includes identifying subsets of cells within a population of cells to be analysed that express a specific biomarker or specific biomarker combinations. A biomarker may be a cell surface antigen or may be an intracellular molecule. To identify subsets of cells, one application of flow cytometry may involve performing multi-coloured analysis using antibodies each of which targets biomarker of interest (e.g., according to the examples disclosed herein). Single cells are transferred in the flow cytometer through a stream of fluid and passed by a light source which may activate a fluorescent antibody bound to a cell flowing past the light source. Fluorescent light emitted by an antibody may be detected by an electronic detection apparatus within the flow cytometer that in turn relays this information computationally through creation of gates/gating that classifies cells that are expressing a biomarker and those that are not. In some examples, gating is performed using FlowJo software, for example, FlowJo v10.8.1 software, although other gating software may be used. Typically, a negative control, such as an antibody for a biomarker that is not expressed on the population of cells being analysed is used to set the gating for cells that will be classified as not expressing a biomarker. The set gating is then used for the rest of the antibody panel for the biomarkers of interest. The information is then visualised to correlate the amount of detected fluorescence with the number of cells that are expressing the biomarker of interest and the amount of the biomarker being expressed by those cells. This technique provides a fast, sensitive and high-throughput analysis of a population of cells. Flow cytometry may be used to profile the frequency and intensity of biomarkers expressed by T cells.

The biomarkers to be detected are biomarkers associated with T cell activation, exhaustion, regulatory and memory differentiation. Detection of a biomarker or a specific combination of biomarkers can be used to determine the strength, frequency and type of immune activation. Therefore, in embodiments where flow, or spectral or time or flight or any form of cytometry is used to detect the presence or absence of a presence of biomarkers, the detecting comprises detecting the signal of one or more fluorescently labelled antibodies that bind to the panel of biomarkers of interest. For example, when the panel or biomarkers includes (i) Ki67− the detecting may comprise detecting the signal of a fluorescently labelled Anti-Ki67 antibody, and/or (ii) CD39, the detecting may comprise detecting the signal of a fluorescently labelled Anti-CD39 antibody, and/or (iii) for FoxP3, the detecting may comprise detecting the signal of a fluorescently labelled Anti-FoxP3 antibody. The presence and/or absence of the other named biomarkers herein, when used in the panel of biomarkers, can also be determined by similarly detecting the signal of a fluorescent labelled antibody targeted against the specific biomarker of interest.

Cytometry also includes spectral cytometry or spectral flow cytometry a technique in which an emission spectrum of multiple fluorescing molecules may be captured by a set of detectors across a defined wavelength range. The fluorescence spectrum can be recognized, recorded as a spectral signature, and used as a reference in multicolour applications.

Cytometry also includes mass cytometry which is a variation of flow cytometry in which antibodies are labelled with heavy metal isotopes rather than fluorochromes. The isotopes are analysed through the cells being ionised and the ions are separated by their mass-to-charge ratio and detected as an electrical signal at the terminal gate of the spectrometer. The readout is typically by time-of-flight mass spectrometry.

In the methods of the present invention the analysis may comprise using at least one of flow cytometry, spectral cytometry and/or mass cytometry.

In some embodiments, the cytometry analysis (e.g., the flow cytometry analysis) comprises using computational clustering for a particular subpopulation of T cells and/or T cells which express or which do not express one or more particular biomarkers of interest. Example computational clustering strategies are described in further detail in the Examples section herein. The computational clustering strategies described herein are merely an example and the person skilled in the art would be aware of the various software packages and protocols that could be used to analyse the cytometry data and obtain the same biological result.

In some embodiments, the cytometry analysis (e.g., the flow cytometry analysis) comprises gating for a particular subpopulation of T cells and/or T cells which express or which do not express one or more particular biomarkers of interest. The terms “gating” or “gates”, as used herein, generally refers to the use of software that is associated with flow cytometry, such as manually drawing two-dimensional gates with a mouse on a computer screen, based on the density contour lines that are provided by software tools. Gates are typically used to distinguish between cells that are expressing a biomarker of interest and those that are not. The cells falling in a gate may be selected (gated in) or excluded (gated out) and the process may be repeated for different two-dimensional (or higher) projections of the gated cells (e.g., two-dimensional dot plots), thus resulting in a sequence of gates that describe subpopulations of the multivariate flow cytometry data. Specific gating strategies are described in further detail in the Examples section herein. The specific gating strategies described herein are merely an example and the person skilled in the art would be aware of the various software packages and protocols that could be used to analyse the flow cytometry data and obtain the same biological result. In some examples, gating (i.e., manual gating) is performed using FlowJo software, for example, FlowJo v10.8.1 software, although other gating software may be used.

A biomarker as used herein, is as a phenotypic biomarker that reflects the activation or differentiation state of a T cell in human blood. Biomarkers that may be detected in the methods of the present invention can include (i) Ki67 and CD39, and/or (ii) FoxP3, optionally in combination with other biomarkers listed herein. The methods detect the presence or absence of a panel of biomarkers comprising (i) Ki67 and CD39 and/or (ii) FoxP3, optionally in combination with other biomarkers listed herein. Hence the panel of biomarkers of the present invention comprises (i) Ki67 and CD39 and/or (ii) FoxP3, optionally in combination with other biomarkers listed herein.

An activated and/or exhausted T cell may express CD39 and Ki67 (CD39+ and Ki67+ T cells). Detection of expression of CD39 and Ki67 can be considered to indicate a T cell that is activated and/or exhausted (i.e., in embodiments of the first aspect, and in the second, third and fourth aspects)

An activated and/or exhausted T cell may also be a CD4 Regulatory T cell (i.e., in embodiments of the first aspect and in the fifth aspects). A CD4 Regulatory T cell expresses the biomarker FoxP3. In some embodiments, the CD4 Regulatory T cell also expresses CD39 (i.e., in combination with FoxP3). Detection of FoxP3 expression (and preferably in combination with CD39) can indicate a CD4 T cell that is activated and/or exhausted. A CD4 T cell that expresses FoxP3 is otherwise referred to as a T regulatory cell herein. In some embodiments, the CD4 Regulatory T cell may express FoxP3, Ki67 and CD39. In some embodiments, the CD4 Regulatory T cell may express FoxP3, CD39, and may lack CD45RA.

A naïve and/or resting T cell does not express CD39 or Ki67 (CD39− and Ki67− cells). Lack of detection of expression is CD39 and Ki67 is considered to indicate a T cell is naïve and/or resting.

T cells which are not activated and/or exhausted do not express one or both of CD39 or Ki67 (CD39− and Ki67+ T cells, CD39+ and Ki67− T cells, or CD39− and Ki67− T cells). Lack of detection of expression of one or both of CD39 or Ki67 is considered to indicate a T cell that is not an activated and/or exhausted T cell.

In this disclosure reference to a T cell not expressing a biomarker marker can refer a lack of detection of expression of that biomarker.

The panel of biomarkers may comprise further biomarkers. The panel of biomarkers may include one or more further biomarkers selected from CD45RA, CCR7, PD-1, CD57 or CD38, (i.e., in addition to Ki67, CD39 and/or FoxP3). These biomarkers are considered to further assist in distinguishing between activated and/or exhausted T cells and naïve and/or resting T cells. FOXP3 can be used to select for regulatory T cells (Tregs).

A panel of biomarkers may comprise Ki67, CD39 and FOXP3. In some embodiments, the activated and/or exhausted cells express Ki67 and CD39 and FoxP3.

A panel of biomarkers may comprise Ki67, CD39 and CD45RA In some embodiments, the activated and/or exhausted cells express Ki67 and CD39 but may or may not express CD45RA.

A panel of biomarkers may comprise Ki67, CD39 and CCR7 In some embodiments, the activated and/or exhausted cells express Ki67 and CD39 but may or may not express CCR7.

A panel of biomarkers may comprise Ki67, CD39 and PD-1 In some embodiments, the activated and/or exhausted cells express Ki67 and CD39 but may or may not express PD-1

A panel of biomarkers may comprise Ki67, CD39 and CD57 In some embodiments, the activated and/or exhausted cells express Ki67 and CD39 but may or may not express CD57.

A panel of biomarkers may comprise Ki67, CD39 and CD38 In some embodiments, the activated and/or exhausted cells express Ki67 and CD39 but may or may not express CD38.

A panel of biomarkers may comprise Ki67, CD39, CD45RA and CCR7 In some embodiments, the activated and/or exhausted cells express Ki67 and CD39 but may or may not express CD45RA and may or may not express CCR7.

A panel of biomarkers may comprise Ki67, CD39, CD45RA, CCR7, and PD-1 In some embodiments, the activated and/or exhausted cells express Ki67 and CD39 but may or may not express CD45RA, may or may not express CCR7, and may or may not express PD-1.

A panel of biomarkers may comprise Ki67, CD39, CD45RA, CCR7, PD-1 and CD57 In some embodiments, the activated and/or exhausted cells express Ki67 and CD39 but may or may not express CD45RA, may or may not express CCR7, may or may not express PD-1 and may or may not express CD57.

A panel of biomarkers may comprise Ki67, CD39, CD45RA, CCR7, PD-1, CD57 and CD38. In some embodiments, the activated and/or exhausted cells express Ki67 and CD39 but may or may not express CD45RA, may or may not express CCR7, may or may not express PD-1, may or may not express CD57 and may or may not express CD38.

A panel of biomarkers may comprise Ki67, CD39, CD45RA, CCR7, CD57, and CD38. In some embodiments, the activated and/or exhausted cells express Ki67 and CD39 but may or may not express CD45RA, may or may not express CCR7, may or may not express CD57 and may or may not express CD38.

A panel of biomarkers may comprise Ki67, CD39, CD45RA, PD-1 and CD57. In some embodiments, the activated and/or exhausted cells express Ki67 and CD39 but may or may not express CD45RA, may or may not express PD-1 and may or may not express CD-57.

In some embodiments, the activated and/or exhausted cells express Ki67 and CD39 but do not express CD45RA. In some embodiments, the activated and/or exhausted cells express Ki67 and CD39 but do not express PD-1. In some embodiments, the activated and/or exhausted cells express Ki67 and CD39 but do not express CCR7. In some embodiments, the activated and/or exhausted cells express Ki67 and CD39 but do not express CD45RA, CCR7 and PD-1. The activated and/or exhausted T cells may be CD8+ T cells, or CD8+CD3+ T cells.

The above panel of biomarkers may be combined with (i) CD3, CD4 or CD8, or (ii) CD3 and CD4 or (iii) CD3 and CD8. The presence of CD3 identifies live T-cells or CD3+ T cells. The presence of CD4 identifies CD4 T cells, i.e., CD4+ T cells or T helper cells. The presence of CD8 identifies CD8 T cells, i.e., cytotoxic T cells or CD8+ T cells.

A panel of biomarkers may comprise FoxP3, or FoxP3 in combination with CD4. In some embodiments, the activated and/or exhausted cells are CD4 T cells that express FoxP3.

A panel of biomarkers may comprise FoxP3 and CD39, or FoxP3 in combination with CD4 and CD39. In some embodiments, the activated and/or exhausted cells are CD4 T cells that express FoxP3 and CD39.

A panel of biomarkers may comprise FoxP3, CD39 and Ki67, or FoxP3, CD39 and Ki67 and CD4. In some embodiments, the activated and/or exhausted cells are CD4 T cells that express FoxP3, CD39 and Ki67.

A panel of biomarkers may comprise FoxP3, CD39 and CD45RA, or FoxP3, CD4, CD39 and CD45RA. In some embodiments, the activated and/or exhausted cells are CD4 T cells that express FoxP3, CD39 and do not express CD45RA. The above panel of biomarkers may be combined with CD3. The panel of biomarkers may include one or more further biomarkers selected from CCR7, PD-1, CD57 or CD38.

Ki67 generally refers to the proliferation biomarker protein Ki-67, encoded by the MK167 gene and is widely used as a biomarker to assess cell proliferation.

CD39 generally refers to Cluster of Differentiation 39, encoded by the ENTPD1 gene and is a ectonucleoside triphosphate diphosphohydrolase.

CD45RA generally refers to the long isoform of the cell-surface tyrosine phosphatase CD45 (lymphocyte common antigen) encoded by the PTPRC gene.

CCR7 generally refers to C-C motif chemokine receptor 7 encoded by the CCR7 gene. It is a member of the G protein-coupled receptor family.

PD-1 (also known as PD1) generally refers to Programmed cell death protein1, encoded by the PDCD1 gene and is a inhibitory receptor on antigen activated T-cells that plays a critical role in induction and maintenance of immune tolerance to self.

CD38, generally refers to Cluster of Differentiation 38 encoded by the CD38 gene and is a cyclic ADP ribose hydrolase found on the surface of many immune cells, including lymphocytes.

FoxP3 is a member of the forkhead transcription factor family and is a master regulatory of the regulatory pathway in the development and function of regulatory T cells.

The term “hi” as used herein, generally refers to a higher/greater abundance of a particular biomarker relative to other T cell types. The term “lo” as used herein, generally refers to a lower/less abundance of a particular biomarker relative to other T cell types. The term “prolif” as used herein generally refers to a proliferating T cell.

Biomarkers may be measured by directly conjugated antibodies which are available from multiple commercial sources. Alternatively, the skilled person may use a primary antibody specific to the biomarker and a secondary antibody to detect the biomarker indirectly. Antibodies may be used to stain peripheral blood mononuclear cells (PBMCs) isolated from whole blood by centrifugation. Staining may also be performed on whole blood. The biomarker profiles (the frequency of cells expressing the biomarker) can be determined by cytometry using instrumentation and software known to the person skilled in the art. Specific antibodies to detect biomarkers of T cell activation or proliferation state may be used. These antibodies may be:
1. Anti-CD39 (to identify activated and exhausted T cells [Tex], tumour reactive T cells)
2. Anti-Ki67 (to identify proliferating T cells)
3. Anti-FoxP3 (identify the biomarker FoxP3, which can be used to identify CD4+T regulatory cells)

As described above additional biomarkers may be detected and methods may use one or more the following antibodies:
4. Anti-CD4, to identify the major CD4+ T cell lineage
5. Anti-CD8, to identify the major CD8+ T cell lineage
4. Anti-CD57 (to identify terminally differentiated T cells)
5. Anti-CD38 (to identify activated and exhausted T cells)
6. Anti-CD45RA (to identify Naïve and terminally differentiated T cells)
7. Anti-PD-1 (to identify activated and exhausted T cells)
8. Anti-CCR7 (to identify naïve and early differentiated memory cells).

In some methods, a preliminary step of identifying live T cells for analysis may be performed. This step may involve antibodies/dyes for 2 biomarkers as follows:
9. A viability dye (e.g. fixable live/dead, which can be obtained from Thermofisher) to exclude dead cells
10. Anti-CD3, to identify T cells.

These are general purpose biomarkers of which the skilled person is aware. An alternative to using CD3 is available to the skilled person which could be to use a T cell enrichment or purification kit and magnetic selection including a CD3 or CD4 or CD8 negative or positive selection kit containing a cocktail of biotinylated or otherwise conjugated antibodies specific to CD3 or CD4 or CD8 or multiple biomarkers on blood cells excluding CD3, CD4 or CD8. Such kits may also be used in flow cytometry, when co-stained with streptavidin or secondary antibodies containing fluorophores.

In some methods, the analysis may be performed on CD4 T cells or CD8 T cells. In some embodiments, prior to analysis, a preliminary step of identifying CD4 T cells or CD8 T cells may be performed, e.g., using an antibody specific to CD4 or CD8 respectively. This may be performed using flow cytometry.

In some methods, the analysis may be performed on a T cell subtype or subpopulation.

In some embodiments, the T cell subtype or subpopulation may be regulatory T cells. Regulatory T cells express the FoxP3 biomarker. In some embodiments, prior to analysis, a preliminary step of identifying regulatory T cells may be performed, e.g., using an antibody specific to the biomarkers FoxP3. This analysis is performed on CD4 T cells. In some embodiments, prior to analysis, a preliminary step of identify CD4+ T cells may be performed, e.g., using an antibody specific to CD4+ T cells.

In some embodiments, the T cell subtype or subpopulation may be an effector memory cell (Teffm). Effector memory T cells may be absent of CCD7, CD45RA and/or PD1 biomarkers. In some embodiments, prior to analysis, a preliminary step of identifying an effector memory cell may be performed, e.g., using one or more antibodies specific to CCD7, CD45RA, and/or PD1. This analysis may be performed in CD8+ T cells, or CD3+CD8+ T cells (e.g., using an antibody specific to CD8 T cells or CD3+CD8+ T cells respectively).

It will be understood that the term “T cell differentiation index”/“TEDI”, as used herein, provides a means to represent the data obtained herein to aid in interpretation of the scope of the invention and is not intended to limit the scope of the invention. The person skilled in the art will understand that such a methodology is an in-house term that is specific to the inventors and that various methods and techniques discussed herein can be performed using a variety of different apparatus, assay conditions, hardware and software components that may result in different numerical values obtained, but which equate to the same biological results to the examples provided herein; that the ratio of exhausted/activated T cells to naïve/resting T cells is higher in subjects with progressive, pre-cancerous lesion or nodule or in the presence of a malignant tumour relative to a subject with an indolent or regressive or non-progressing or low grade disease or no disease or a benign solid tumour.

The term “T cell differentiation index”/“TEDI”, as used herein, generally refers to the ratio of activated and/or exhausted T cells:naïve and/or resting T cells within a subject which can be represented numerically in the form of an index when a subject's results are compared against the average ratio of activated and/or exhausted T cells:naïve and/or resting T cells obtained from a plurality of patients known to have progressing or high-grade pre-invasive lesions or known to have a cancer to infer the risk that the subject has a progressing or high-grade pre-invasive lesion or nodules or having a solid malignant tumour. A subject's results may also be compared against their own previously determined ratio to indicate whether that subject is at risk of having a progressing or high-grade pre-invasive lesion or nodules or having a solid malignant tumour.

The methods of the present invention are performed on a blood sample obtained from a subject and hence occur in vitro. The methods can use whole blood. The methods can use PBMCs.

The present invention also provides a kit comprising a set of lyophilised antibodies or a fragment thereof, comprising antibodies binding to (i) CD39 and Ki67 and/or (ii) FoxP3 and CD4. Such a kit may be used in or to perform the methods of the present invention. In embodiments, the kit may comprise one or more further lyophilised antibodies selected from antibodies binding to one or more biomarkers selected from CD45RA, CCR7, PD-1, CD57 or CD38. The kit may be used in a method for determining whether a subject is at risk for having a progressing or high-grade pre-invasive lesion or nodule or having a solid tumour.

The present invention also provides a device comprising means for receiving a sample of blood and contacting the sample with a set of lyophilised antibodies or a fragment thereof, comprising antibodies binding to (i) CD39 and Ki67 and/or (ii) FoxP3 and CD4. Such a device may be used in or to perform the methods of the present invention. The device may be used in a method for determining whether a subject is at risk for having a progressing or high-grade pre-invasive lesion or nodule or having a solid tumour

It will be understood that the present invention may be of use in providing information on whether a subject is at risk for having a progressing or high-grade pre-invasive lesion or nodule or having a solid malignant tumour. The methods of the present invention can also be implemented for valuable healthcare outcomes, examples by way of illustration, are set out below.

### A) Multi-Cancer Screening

At risk populations for any cancer to receive blood test (e.g. those with hereditary predispositions due to germline mutations). A positive result would trigger a confirmatory second test. A positive second test then triggers clinical follow up for relevant cancer type. The invention therefore provides a valuable tool which works on a variety of cancers, even when they come from different causes.

### B) Screening for Lung Cancer or Pre-Invasive Lung Neoplasia.

At-risk populations such as smokers or ex-smokers over 50 years of age to receive blood tests. A positive result would trigger a confirmatory second test. A positive second test then triggers sputum cytology, bronchoscopy and LDCT screen.

### C) Screening for Renal Cell Carcinoma.

At risk populations to receive blood test. A positive result would trigger a confirmatory second test. A positive second test then triggers clinical follow up including ultrasound, MRI X-ray or CT scan.

### D) Personalised Dynamic Immune Monitoring

An initial negative test is used to calibrate a personalised baseline value. A statistically significant increase in ratio of activated and/or exhausted T cells:naïve and/or resting T cells in any future test triggers a follow up test, where a positive result triggers further clinical evaluation for cancer types relevant to the demographic/subject.

### E) Healthy Adult Population Level Screening (Mass Testing)

Adults above the age of 40 can be screened, a positive result would trigger a confirmatory second test. A positive second test then triggers broad clinical follow up to screen for cancers of major incidence in the demographic. A negative test is used to establish an individual's personalised baseline.

### F) Detection of Cancer Recurrence

Subjects with a history of cancer may be screened. Scores obtained with methods of the present invention decrease post resection, suggesting that timepoints early after tumour removal/eradication may be suitable to establish a baseline. Statistically significant increases trigger a second test and/or clinical follow up. This would be to detect either minimal residual disease and or recurrence.

### G) Inclusion in Standard Clinical Blood Work

The invention disclosed herein could be incorporated into standard clinical blood work alongside tests such as ESR/CRP/white blood cell count or full blood count.

### Methods of Treatment

Also disclosed herein is a method of treating a subject determined to be at risk for having a progressing or high-grade pre-invasive lesion, nodule or small mass, or having a solid malignant tumour, wherein the method comprises:
determining whether a subject is at risk for having a progressing or high-grade pre-invasive lesion, nodule or small mass, or having a solid malignant tumour, the method comprising:
determining a ratio of activated and/or exhausted T cells:T cells which are not activated and/or exhausted T cells in a sample of blood obtained from the subject,
wherein the determining comprises analysing T cells using cytometry to detect the presence or absence of a panel of biomarkers comprising Ki67 and CD39, and
wherein the subject is at risk if
the ratio of activated and/or exhausted T cells:T cells which are not activated and/or exhausted is equal to or greater than
a ratio of activated and/or exhausted T cells:T cells which are not activated and/or exhausted of a comparison subject, or
an average ratio of activated and/or exhausted T cells:T cells which are not activated and/or exhausted of a plurality of comparison subjects, and
providing treatment to said subject, if said subject is determined to be at risk.

Also disclosed herein is a method of treating a subject determined to be at risk for having a progressing or high-grade pre-invasive lesion, nodule or small mass, or having a solid malignant tumour, wherein the method comprises:
determining whether a subject is at risk for having a progressing or high-grade pre-invasive lesion, nodule or small mass, or having a solid malignant tumour, the method comprising:
determining the proportion of activated and/or exhausted T cells as a percentage of T cells in the sample of blood of a subject, wherein the determining comprises analysing T cells using cytometry to detect either (i) the presence of FoxP3, and/or (ii) the presence of Ki67 and CD39 and
wherein the subject is at risk if
the proportion of activated and/or exhausted T cells is higher than the proportion of activated and/or exhausted T cells in a comparison subject, or
the proportion of activated and/or exhausted T cells is higher than the average proportion of activated and/or exhausted T cells in a plurality of comparison subjects,
and
providing treatment to said subject, if said subject is determined to be at risk.

In some embodiments, the treating may comprise administering an anti-cancer therapeutic. In some embodiments, the treating may comprise administering a therapeutic suitable for treating pre-invasive neoplasia and/or a high grade pre-invasive lesion, nodule or small mass. In some embodiments, the treating may comprise administering a therapeutic suitable for treating a solid malignant tumour, for example, a stage I solid malignant tumour. In some embodiments, the treating may comprise electrocautery, argon plasma coagulation (APC), cryotherapy and photodynamic therapy (PDT). The latter are all minimally invasive treatment options that may be used for the treatment of high-grade pre-invasive lesions, as described in Daniels et al; Ther. Adv. Med Oncol. 2013 July; 5(4); 235-248.

Also disclosed herein is an anti-cancer therapeutic for use in a method of treating a subject determined to be at risk for having a progressing or high-grade pre-invasive lesion, nodule or small mass, or having a solid malignant tumour, wherein the method comprises:
determining whether a subject is at risk for having a progressing or high-grade pre-invasive lesion, nodule or small mass, or having a solid malignant tumour, the method comprising:
determining a ratio of activated and/or exhausted T cells:T cells which are not activated and/or exhausted T cells in a sample of blood obtained from the subject,
wherein the determining comprises analysing T cells using cytometry to detect the presence or absence of a panel of biomarkers comprising Ki67 and CD39, and
wherein the subject is at risk if
the ratio of activated and/or exhausted T cells:T cells which are not activated and/or exhausted is equal to or greater than
a ratio of activated and/or exhausted T cells:T cells which are not activated and/or exhausted of a comparison subject, or
an average ratio of activated and/or exhausted T cells:T cells which are not activated and/or exhausted of a plurality of comparison subjects, and
administering the anti-cancer therapeutic to said subject, if said subject is determined to be at risk.

Also disclosed herein is an anti-cancer therapeutic for use in a method of treatment of a subject determined to be at risk for having a progressing or high-grade pre-invasive lesion, nodule or small mass, or having a solid malignant tumour, wherein the method comprises:
determining whether a subject is at risk for having a progressing or high-grade pre-invasive lesion, nodule or small mass, or having a solid malignant tumour, the method comprising:
determining the proportion of activated and/or exhausted T cells as a percentage of T cells in the sample of blood of a subject, wherein the determining comprises analysing T cells using cytometry to detect either (i) the presence of FoxP3, and/or (ii) the presence of Ki67 and CD39 and
wherein the subject is at risk if
the proportion of activated and/or exhausted T cells is higher than the proportion of activated and/or exhausted T cells in a comparison subject, or
the proportion of activated and/or exhausted T cells is higher than the average proportion of activated and/or exhausted T cells in a plurality of comparison subjects,
and
administering the anti-cancer therapeutic, if said subject is determined to be at risk.

### EXAMPLES

In this section, Examples are described for analysing T cells isolated from a subject. The methods in the Examples provided herein for analysing T cells isolated from a subject can be implemented using a variety of methodologies, including using a variety of apparatus, hardware and software components that include (but are not limited to) different cytometry devices, assay conditions, computational hardware and analysis software. It will be understood to the person skilled in the art that in general, the methods provided herein in the Examples are merely an example. The person skilled in the art will know that that the various steps and techniques discussed herein can be performed using a variety of different apparatus, assay conditions, computational hardware and software components that may result in different numerical values obtained, but which equate to the same biological results to the Examples provided herein. It will be understood to the person skilled in the art that in general, the methodologies and results provided herein in the below Examples are not intended to limit the scope of the invention.

TABLE 1
List of antibodies used in Examples 1-3
Samples were acquired on FACSymphony A5 High-Parameter
Cell Analyser from BID Biosciences.
Phenotyping Panel: Fluorochromes and Antibodies
(Biomarkers are surface biomarkers unless otherwise stated)

| Channel | Target | Clone | Supplier | Cat# |
|---|---|---|---|---|
| BUV395 | Ki67 | B56 | BD | 564071 |
| | (intracellular biomarker) | | Biosciences | |
| BUV496 | CD8 | RPA-T8 | BD | 741199 |
| | | | Biosciences | |
| BUV563 | CD45RA | HI100 | BD | 612926 |
| | | | Biosciences | |
| BUV615 | CD4 | OKT4 | BD | 750975 |
| | | | Bioscience | |
| BUV661 | CD101 | V7.1 | BD | 750530 |
| | | | Bioscience | |
| BUV737 | CD38 | HB-7 | BD | 741902 |
| | | | Biosciences | |
| BUV805 | CD103 | Ber-ACT-8 | BD | 748501 |
| | | | Biosciences | |
| BV421 | FOXP3 | 206D | Biolegend | 320124 |
| | (intracellular biomarker) | | | |
| BV480 | Live/Dead Fixable | N/A | ThermoFisher | L34966 |
| | Aqua | | | |
| BV605 | CD57 | QA17A04 | Biolegend | 393304 |
| BV650 | CCR7 | G043H7 | Biolegend | 353234 |
| | (chemokine receptor) | | | |
| BV711 | CD39 | A1 | Biolegend | 328228 |
| BV750 | CD27 | O323 | Biolegend | 302850 |
| BV785 | TIM3 | F38-2E2 | Biolegend | 345032 |
| BB515 | PD-1 | EH12.1 | BD | 594494 |
| | | | Biosciences | |
| PerCP-eFluor | EOMES | WD1928 | eBioscience | 46-4877-42 |
| 710 | (intracellular biomarker) | | | |
| PE | TCF7 | 7F11A10 | Biolegend | 655208 |
| | (intracellular biomarker) | | | |
| PE-CF594 | Granzyme B | GB11 | BD | 562462 |
| | (intracellular biomarker) | | Biosciences | |
| PE-Cy5 | CXCR4 | 12G5 | Biolegend | 306508 |
| | (chemokine receptor) | | | |
| PE-Cy7 | TIGIT | A15153G | Biolegend | 372714 |
| APC | Tox | REA473 | Miltenyi | 130-120-716 |
| | (intracellular marker) | | Biotec | |
| APC-R700 | HLA-DR | G46-6 | Biolegend | 565127 |
| APC-Fire | CD3 | UCHT1 | Biolegend | 300470 |

### An Example Protocol for Processing of Blood Samples for Flow Cytometry Analysis

Blood samples were collected in Vacutainer EDTA blood collection tubes (BD), PBMCs isolated by gradient centrifugation (750 g for 10 minutes) on Ficoll Paque Plus (GE Healthcare). The interface was washed twice with complete RPMI-1640, resuspended in 90% FBS with 10% DMSO (Sigma) and cryopreserved in liquid nitrogen or in a −180 degrees Celsius freezer system prior to staining. To characterise T cell differentiation profiles of HG vs LG samples cryopreserved PBMCs from were thawed with warm R20 media (RPMI with 20% FBS, L-glutamine, HEPES and Penicillin/Streptomycin) and washed with R10 media containing DNase I grade II 37.5 μg/mL (Roche, 10104159001). The samples were subjected to 20 mins incubation of Live/Dead Fixable Aqua at RT in the dark. Samples were then washed in PBS and resuspended with FACS buffer (PBS+2% FBS+2 mM EDTA) and plated on a 96-well U bottom plate containing Fc Receptor Binding Inhibitor Polyclonal Antibody (Thermo Fisher, 14-9161-73) and surface antibodies for chemokine receptors (CXCR4, CCR7) at room temperature (RT) for 15 mins, followed by another 30 mins incubation with all additional surface antibodies, on ice. Plates were washed twice with FACS buffer and fixed with Foxp3 Transcription Factor Fixation/Permeabilization Concentrate and Diluent solutions (Thermo Fisher, 00-5521-00) for 30 mins at RT. Cells were washed twice with ×1 Permeabilization Buffer (Thermo Fisher, 00-8333) followed by 1 hour incubation with a cocktail of intracellular antibodies at RT. The plates were washed 3 times and resuspended with ×1 Permeabilization Buffer before sample acquisition using FACSymphony (BD Biosciences.

### An Example Protocol for Acquisition and Processing

Samples were acquired using a BD Symphony cytometer according to the manufacturer's instructions using the BD FACS DIVA software. Raw FCS files were exported and files imported into FlowJo. Compensation matrices were calculated in FACS DIVA from single-stain compensation bead controls and optimised in FlowJo. To do so, files from each batch (two in total from consecutive days) were concatenated separately as a reference file and optimised matrices applied to each batch. Dead cells and compensation artefacts were excluded as described previously (NC) and 5000 live CD3+CD4+ or 2000 CD3+CD8+ events were down-sampled and exported per file.

### An Example Protocol for Clustering

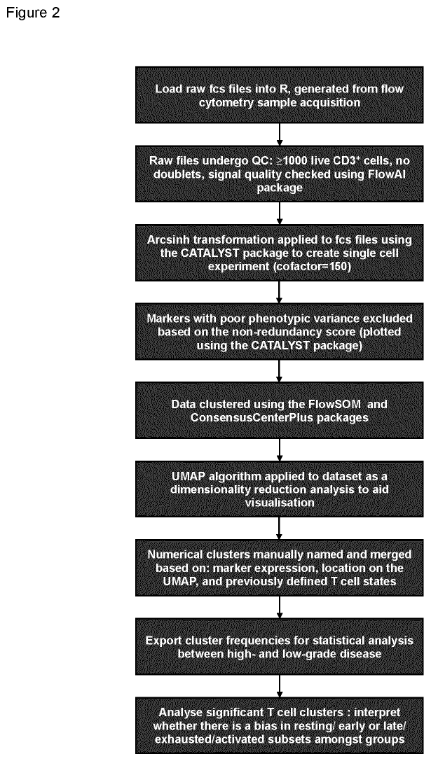

FIG. 2 shows a workflow showing the protocol of how the clustering of the flow cytometry data was completed following the analysis steps outlined.

Only samples with minimum 1000 live CD3+ cells were analysed. FCS files underwent quality control of signal acquisition, assessed by the FlowAI package (v1.24). An Arcsinh transformation was applied to the data, using the prepData function from the CATALYST package (v1.18.1), with the cofactor set to 150. Any biomarkers with a poor contribution to phenotypic variance were excluded pre-clustering. These were determined using the PCA-based non-redundancy score (NRS) as described in the Nowicka et al. pipeline (Nowicka M, Krieg C, Crowell H L et al. CyTOF workflow: differential discovery in high-throughput high-dimensional cytometry datasets [version 4; peer review: 2 approved]. F1000Research 2019, 6:748 (https://doi.org/10.12688/f1000research.11622.4). In initial analysis (e.g., of the pre-invasive data), FoxP3, CD103, Tim3, CXCR4, TIGIT, were all excluded from clustering (live/dead, CD3, CD8 and CD4 were excluded as FCS files only included cells pre-gated on live CD4 or CD8 T cells). In subsequent analysis, FoxP3 was included in the clustering panel.

Data was clustered using the FlowSOM package (v2.2.0) onto a 15×15 node square self-organising map (SOM). Nodes were clustered by the ConsensusCenterPlus package (v1.58.0), as described by Nowicka et al. The UMAP algorithm was applied as a dimension reduction analysis to better understand the phenotypic relevance between individual clusters. The resulting clusters were manually merged based on biomarker expression similarity, occupation of space on the UMAP, and previously defined T cell states. 16 clusters of CD4 T cells and 15 clusters of CD8 T cells were resolved. All computational packages were sourced from https://cran.r-project.org/and https://www.bioconductor.org/.

TABLE 2
A list of T cell clusters that were differentially abundant between high
grade or low grade pre-invasive lesion or nodule.
Clusters found to be enriched in high-grade were found to express biomarkers of
activation and late differentiation, proliferation or exhaustion, in particular the biomarkers
CD39, known to be expressed on tumour specific T cells, and the marker Ki67 which
denotes that cells are actively proliferating. TEDI scores were generated by dividing high-
grade-associated clusters by the low grade associated clusters within each lineage (e.g.,
sum of high grade clusters/sum of low grade clusters* for CD4 = CD4.TEDI) to develop
a metric that would have the highest discriminatory power, which reflects the state of
differentiation within that lineage. The same was completed for CD8 T cells and all T
cells combining CD4 and CD8.

| | | | | Biological | Enrichment | |
|---|---|---|---|---|---|---|
| Subset | Full name | Lineage | Biomarkers | relevance | in PID* | Identified by |
| Tex.Prolif | Exhausted, | CD8 | PD-1, | Exhaustion, | High | Unsupervised |
| | proliferating | | Ki67, | activation | grade | clustering |
| | CD8 T cell | | CD39 | | | |
| TEMRA.Act | Terminally | CD8 | CD39, | Terminal | High | Unsupervised |
| | differentiated | | Ki67 | differentiation, | grade | clustering |
| | effector | | CD45RA, | activation | | |
| | memory | | CD57 | | | |
| | cells (RA) | | | | | |
| | that are | | | | | |
| | activated | | | | | |
| Naive | Naïve | CD8 | CD45RA, | Resting, | Low | Unsupervised |
| | CD8 T | | CCR7 | unstimulated, | grade | clustering |
| | cells | | | non-antigen | | |
| | | | | experienced | | |
| TEMRA.Rest | Terminally | CD8 | CD4RA | Resting | Low | Unsupervised |
| | differentiated | | CCR7− | terminally | grade | clustering |
| | effector | | CD57− | differentiated | | |
| | memory | | | memory T cells | | |
| | cells (RA) | | | | | |
| | that are | | | | | |
| | resting | | | | | |
| Treg.prolif.PD-1 | Regulatory | CD4 | CD39, | Antigen- | High | Unsupervised |
| | T cells | | Ki67, | Activated | grade | clustering |
| | that are | | PD-1, | regulatory T | | |
| | proliferating | | | cells | | |
| | and express | | | | | |
| | PD-1 | | | | | |
| TEM | Effector | CD4 | CD39, | Effector | High | Unsupervised |
| | memory | | Ki67, | memory | grade | clustering |
| | CD4 T cells | | CCR7− | CD4 T cells | | |
| | | | CD45RA− | | | |
| Treg | Regulatory | CD4 | CD39, | Regulatory | High | Unsupervised |
| | T cells | | Ki67, | T cells | grade | clustering |
| | | | | suppress | | |
| | | | | immunity | | |
| | | | | and play a | | |
| | | | | role in | | |
| | | | | cancer | | |
| | | | | promotion | | |
| TPEX | Progenitor | CD4 | CD39, | Early | High | Unsupervised |
| | exhausted | | Ki67, | version of | grade | clustering |
| | CD4 T | | PD-1 | exhausted | | |
| | cells | | | CD4 T cells | | |
| Treg.prolif | Proliferating | CD4 | CD39, | Proliferating | High | Unsupervised |
| | regulatory | | Ki67hi, | regulatory T | grade | clustering |
| | T cells | | PD-1 | cells | | |
| Tem.Prolif | Proliferating | CD4 | CD39, | Activated, | High | Unsupervised |
| | effector | | Ki67hi, | dividing late | grade | clustering |
| | memory | | CCR7− | differentiated | | |
| | CD4 T | | CD45RA− | effector | | |
| | cells | | | memory cells | | |
| TCM.CD38 | Central | CD4 | CD39, | An early | High | Unsupervised |
| | memory | | Ki67hi, | activated T | grade | clustering |
| | CD4 T | | CD38, | cell subset | | |
| | cells | | CCR7, | | | |
| | expressing | | CD45A | | | |
| | CD38 | | | | | |
| Tex.Prolif | Exhausted, | CD4 | CD39, | Exhaustion, | High | Unsupervised |
| | proliferating | | Ki67hi, | activation | grade | clustering |
| | CD8 T cell | | PD-1 | | | |
| Cytolytic | Cytolytic | CD4 | CD57 | Terminally | High | Unsupervised |
| | CD4 T cells | | | differentiated | grade | clustering |
| | | | | and cytotoxic | | |
| | | | | CD4 T cells | | |
| Naïve.TCF7neg | Naïve | CD4 | CD45RA+ | A subset of | Low | Unsupervised |
| | CD4 T | | CCR7+ | naïve T cell | grade | clustering |
| | cells that | | CD27+ | | | |
| | have lost | | TCF7− | | | |
| | TCF7 | | | | | |
| | expression | | | | | |
| TCM.rest | Resting | CD4 | CD45RA− | Early | Low | Unsupervised |
| | central | | CCR7+ | differentiated | grade | clustering |
| | memory | | CD38− | resting memory | | |
| | CD4 T | | | cells that are | | |
| | cells | | | unstimulated | | |
| Naïve | Naïve T | CD4 | CD45RA+ | Naïve, | Low | Unsupervised |
| | cells | | CCR7+ | unstimulated | grade | clustering |
| | | | | T cells | | |

### Example 1

In Example 1, the inventors used the example protocols set out above.

TABLE 3
Clinical variables of patients used to generate data in Example 1.

Total

| Patients | 30 | | | |
|---|---|---|---|---|
| Samples | 69 | | Mean 2.3 (1-6) | |
| Age (mean) | 68.5 | | (52-89) | |
| Smoking status | | | | |
| Never | 2 | patients | 3 | samples |
| Ex | 19 | patients | 42 | samples |
| Current | 9 | patients | 24 | samples |
| Pack years (mean) | 46.4 | | (0-120) | |
| Grade | | | | |
| High | 14 | patients | 34 | samples |
| Low | 16 | patients | 35 | samples |

### Analysis of Cluster Distribution in High Versus Low Grade Samples

The frequency of each cluster was expressed as a % of parent (CD4 or CD8) for each of the 66 samples and Mann-Whitney tests were performed in R to compare average cluster frequency between high-grade (HG) vs low-grade (LG) samples for each cluster. Separate rounds of analysis focused on CD4 and CD8 T cell subsets were performed, with multiple correction adjustment via benjamini-hochberg, applying a false discovery rate of 0.05. To account for multiple samples being drawn from individual patients, analysis was repeated using the mean frequency of a given cluster in each patient, using all available samples, yielding similar results.

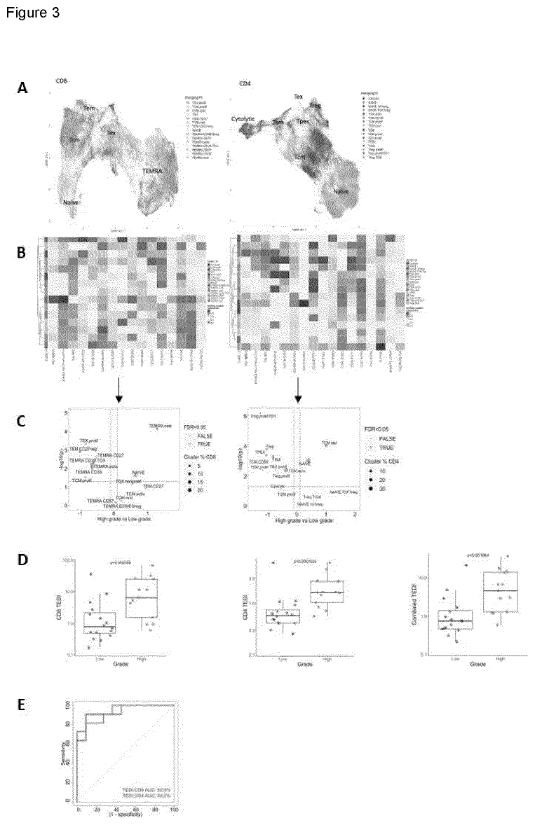

12 (of 16) clusters of CD4 T cell clusters and 4 (of 15) CD8 T cell clusters were significantly differentially abundant in HG vs LG (FDR<0.05 sample level analysis and p<0.05 in patient level analysis). The clusters enriched in HG patients represented late T cell differentiation, activation and exhaustion within both CD4 and CD8 T cells. This shows which clusters of T cells are present across all samples and their relative abundance. Thereby showing that T cells can be classified based on their phenotype into low-grade and high-grade disease. FIG. 3C. The presence of HG (vs LG) lesions is associated with a significantly increased frequency of activated, memory and exhausted CD8 and CD4 T cells and a loss of resting Tcm and naïve cells in the blood (FIG. 3A), This shows that there are multiple types of activated/exhausted and resting/naïve T cell present in the pool of samples. further demonstrated with significantly different expression of CD45RA, CCR7, Ki67, CD39, CD57 and CD38 biomarkers in activated and/or exhausted T cell types compared to naïve and/or resting T cell types (FIG. 3B-C). The ratio of [T cell clusters significantly enriched in high-grade disease samples:T cell clusters significantly enriched in low grade samples] for CD4 (AUC 94.2%) or CD8 (AUC 92.6%) T cells were able to help discriminate patients with HG lesions in ROC analysis, meaning that 92-94% of patients can be correctly classified as having High or Low-grade disease using the invention. FIG. 3D-E.

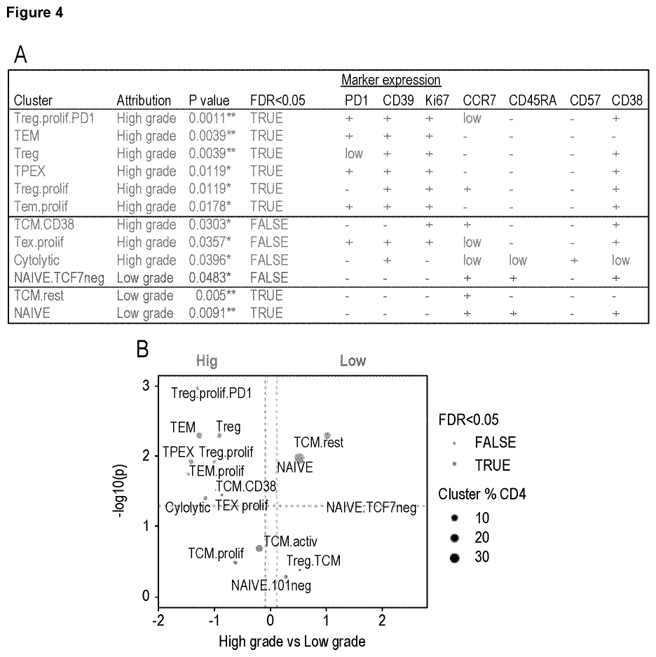

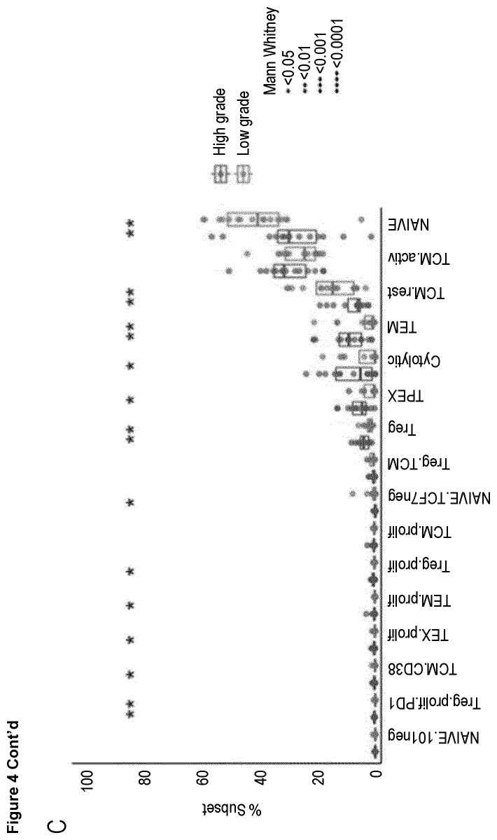

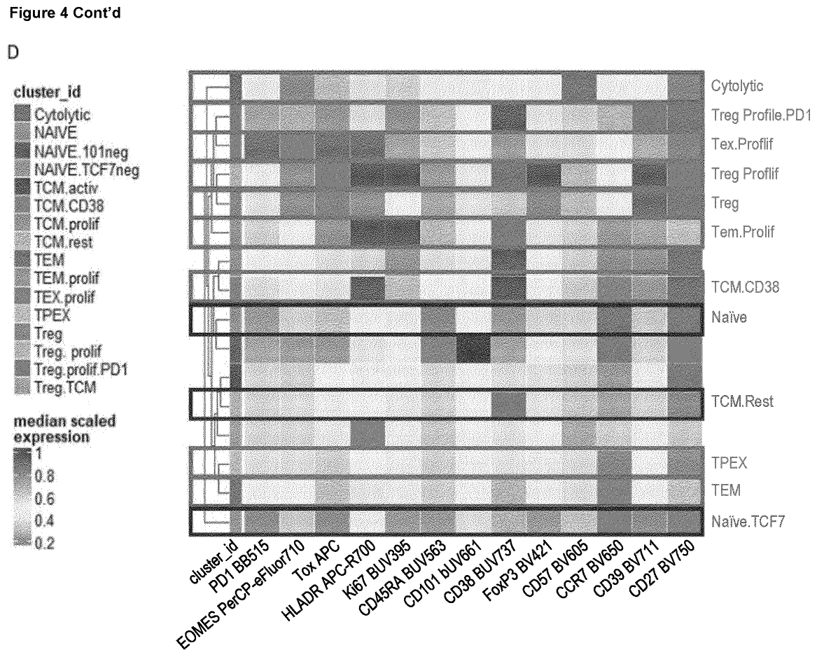

In CD4 T cells 8 of the 9 subsets significantly over-represented in patients with HG disease expressed at least one of the following biomarkers: PD-1 (a biomarker of antigen driven activation and exhaustion; 6/9 HG-clusters) CD39 (a biomarker of exhausted, regulatory and tumour reactive T cells; 8/9 HG-clusters) and Ki67 (an activation biomarker expressed on proliferating T cells; 8/9 HG-clusters). Moreover, CD39 and Ki67 co-expression defined 7 of the 9 T cell subsets enriched in HG disease. HG associated clusters of CD4 T cells also lacked CD45RA and 7 of 9 also lacked high levels of the early differentiation biomarker CCR7. In contrast, all 3 CD4 T cell clusters enriched in LG were devoid of the activation and exhaustion biomarkers PD-1, Ki67, and CD39 (i.e. PD-1−K167−CD39−), and all 3/3 expressed CCR7 and 2/3 expressed CD45RA. This imbalance in CD4 T cell differentiation is indicative of increased antigen exposure with HG disease and suggests that HG vs LG patients can be distinguished using a combination of the biomarkers PD-1, Ki67, CD39, CCR7 and CD45RA. In addition, a single cluster of Cytolytic phenotype CD4 T cells enriched in HG disease that could be identified by unique expression of the terminal differentiation biomarker CD57. FIG. 4. This shows that expression of CD45RA, CCR7, Ki67, CD39, CD57 and CD38 biomarkers were significantly different in activated and/or exhausted T cell types compared to naïve and/or resting T cell types and that these biomarkers can be used to determine the ratio of activated and/or exhausted T cells compared to naïve and/or resting T cells in a subject, thereby distinguishing between high-grade and low-grade disease.

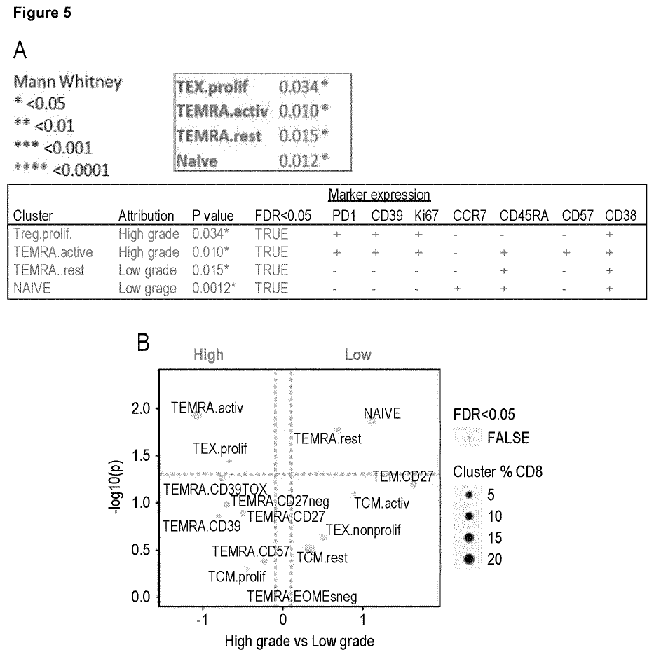

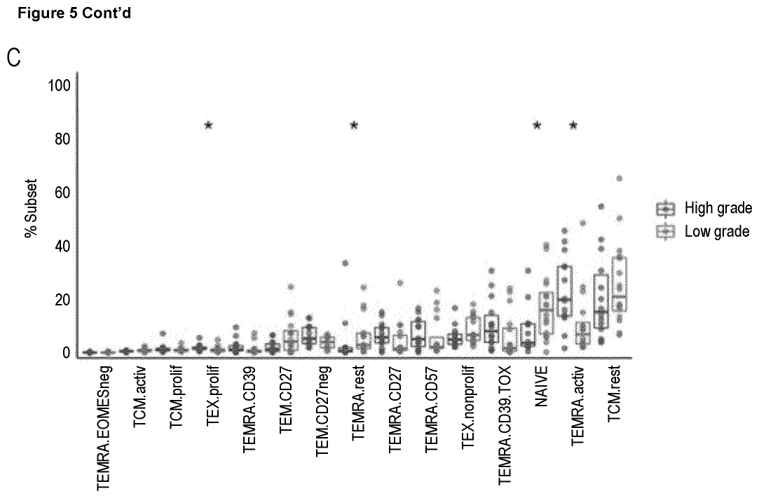

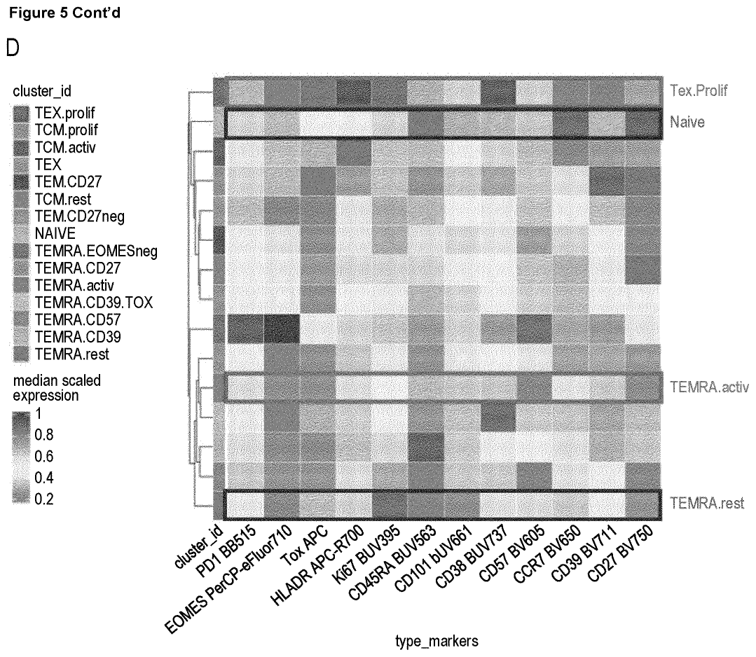

Skewing in T cell differentiation was also observed in CD8 T cells, where two clusters were enriched in HG disease that represent end points of late-stage differentiation and activation in the blood. These 2 clusters once again both co-expressed CD39 and Ki67, but could be distinguished as exhausted (PD-1 high) or terminally differentiated (CD57+CD45RA+) based on additional biomarkers. FIG. 5 As observed in CD4 T cells, CD8 T cell clusters enriched in LG disease were PD-1−CD39−Ki67− indicating a resting state. These clusters were naïve cells known to be enriched in less antigen experienced individuals, and resting memory cells. Identification of these 4 populations from all remaining CD8 T cells via clustering required the same 6 biomarkers as CD4 T cells; namely PD-1, Ki67, CD39, CCR7, CD45RA, CD57. In addition, the biomarker CD38 was required to identify the activated, terminally differentiated population. FIG. 5. This shows that expression of CD45RA, CCR7, Ki67, CD39, CD57 and CD38 biomarkers were significantly different in activated and/or exhausted T cell types compared to naïve and/or resting T cell types and that these biomarkers can be used to determine the ratio of activated and/or exhausted T cells compared to naïve and/or resting T cells in a subject, thereby distinguishing between high-grade and low-grade disease. This data therefore supports measuring systemic T cell differentiation as an innovative strategy for early detection. Specifically, the data show that measuring the ratio of activated, exhausted and late memory T cell subsets:Naïve, resting and earlier differentiated T cells is a novel metric that distinguishes individuals with less pathogenic lesions (LG) from patients with lesions more likely to progress to NSCLC (HG).

### Generation of an Example of a T Cell Early Detection Index (TEDI)

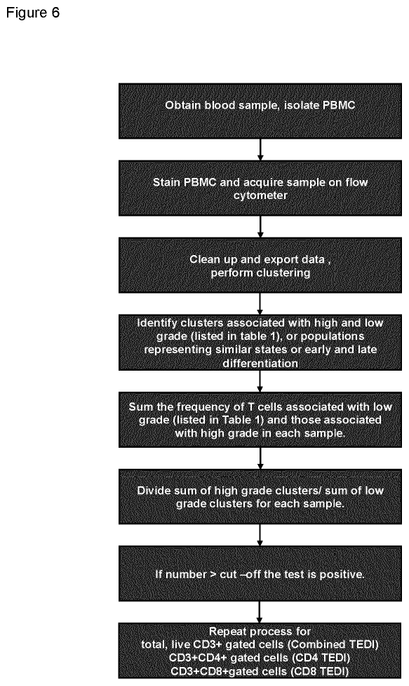

To simplify these results, the change in CD4 or CD8 clusters was converted to a single score referred to as the T cell early detection index (TEDI). To generate TEDI a ratio of [sum freq. of all CD4 T cell clusters enriched in HG disease]/[sum freq. of all clusters enriched in LG disease] was calculated and the process repeated for CD8 T cells, a simple workflow is shown in FIG. 6

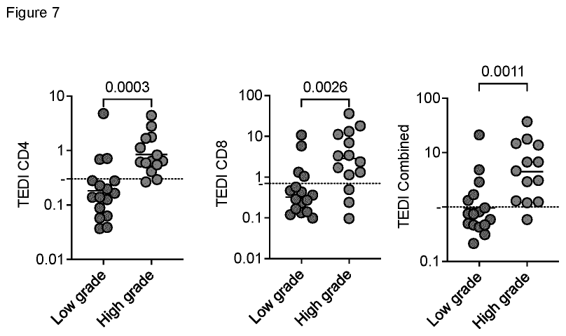

In addition, the mean average of CD4 and CD8 T cell TEDI scores was calculated to generate a Combined TEDI score. Receiver operator characteristic (ROC) curves and calculated the areas under the curve (AUC) were generated for each TEDI score, shown in FIG. 7. This Figure summarises the key results from Example 1, showing that the ratio of exhausted/activated:naïve resting T cells is significantly higher in high vs low grade disease. The same data is also shown in FIG. 3C

TABLE 4
Sensitivity and specificity values with their corresponding
threshold probabilities for ROC analysis
To determine the optimum TEDI cut-off values from the ROC
analysis, all possible sensitivity and specificity values
with their corresponding threshold probabilities were
calculated, as ROC curves represent a trade-off between
the true and false positive rates. The threshold
probability that yielded the highest combined sensitivity
and specificity was selected, known as the Youden Index,
a commonly used method to estimate the optimal cut-off
point for a diagnostic Fluss R, Faraggi D, Reiser B.
Estimation of the Youden Index and its associated cutoff
point. Biom J. 2005 August; 47(4): 458-72. doi:
10.1002/bimj.200410135. PMID: 16161804.
The Youden-determined cut-offs were adjusted to prioritise
sensitivity (reducing false negatives) over specificity
(increasing false positive), yielding threshold
probabilities of the TEDI score required for correct
high-grade patient classification, with the greatest accuracy.
These adjusted cut off values represent the values at which
a test using the TEDI indices would be classified as positive or negative.

| | | | | | Sensitivity |
|---|---|---|---|---|---|
| | | | | Cut-off | Adjusted |
| TEDI | AUC (%) | Sensitivity | Specificity | value | Cut-off |
| CD4 | 94.2 | 0.91 | 0.91 | 0.7054743 | 0.3 |
| | (85.05-100) | (0.73-1) | (0.73-1) | | |
| CD8 | 92.6 | 0.82 | 0.91 | 1.49561 | 0.6 |
| | (81.92-100) | (0.55-1) | (0.73-1) | | |
| Combined | 93.4 | 0.91 | 0.91 | 1.086619 | 1 |
| | (82.6-100) | (0.73-1) | (0.73-1) | | |

### Manual Gating

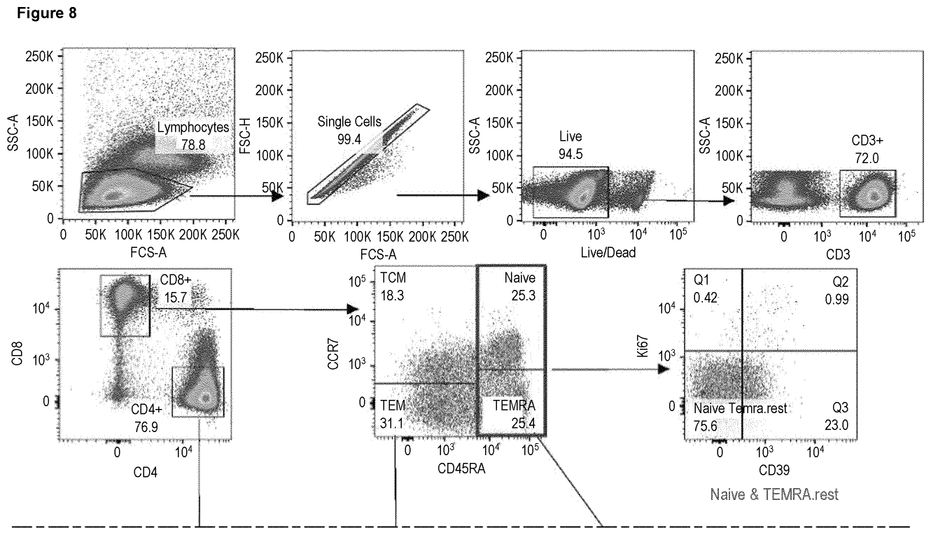

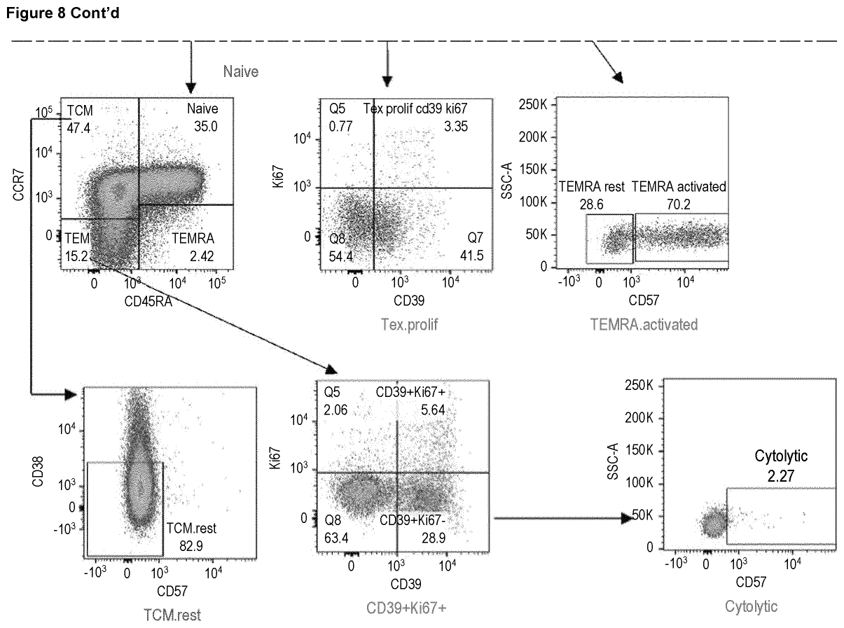

The FlowJo software (BD) was used to manually gate the computationally resolved T cell clusters of interest (those significantly enriched in HG or LG samples after using the mean frequency of each patient's samples). FIG. 8 This shows the person skilled in the art could bypass computational analysis to identify several activated/exhausted and naïve/resting T cell clusters manually using compensated FCS (Flow Cytometry Standard) files in FlowJo software. In this Example, this step is not only necessary to validate output of the unsupervised clustering analysis but also to ensure that equivalent results can be generated with a minimal panel of antibodies. This demonstrates that standard methodologies can be used that do not require computational expertise, development of a TEDI or other similar forms of statistical indexing/classification. This means that a subject's population of T cells can be analysed and where the ratio of that subject's T cells show more activated and/or exhausted T cell's compared to naïve/resting T cells based on detection of a combination of biomarker expression, a subject can be deemed at risk of having a progressing or high-grade pre-invasive lesion or nodule or having a solid tumour. A subject can then be administered to further testing, thereby simplifying clinical implementation. Such standard methodology can be applied to a point of care device (not shown) where a subject applies a blood sample to the device containing a panel of antibody biomarkers for analysis with the subject's T cells and where the combination of biomarkers present on the subject's T cells will provide an indication whether the subject is at risk of having a progressing or high-grade pre-invasive lesion or nodule or having a solid tumour. In preliminary analysis of the pre-invasive data, the results showed that a panel of CD45RA, CCR7, Ki67, CD39, CD57 and CD38 biomarkers can be applied to manually gate the clusters of CD8 and CD4 T cells that were significantly different in high-grade or low-grade disease. FIG. 8.

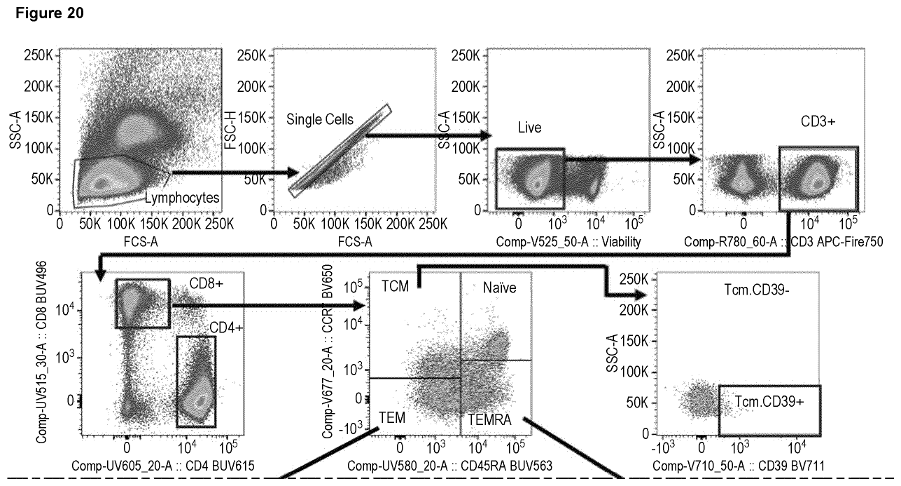

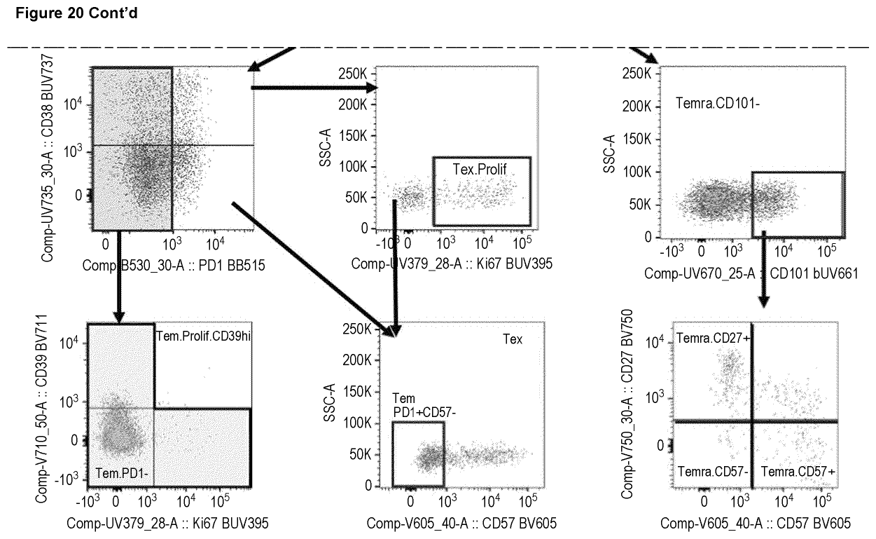

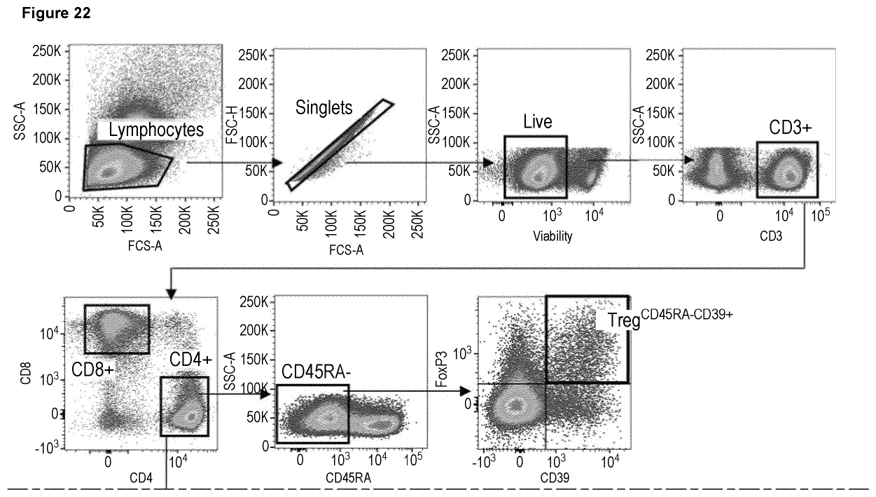

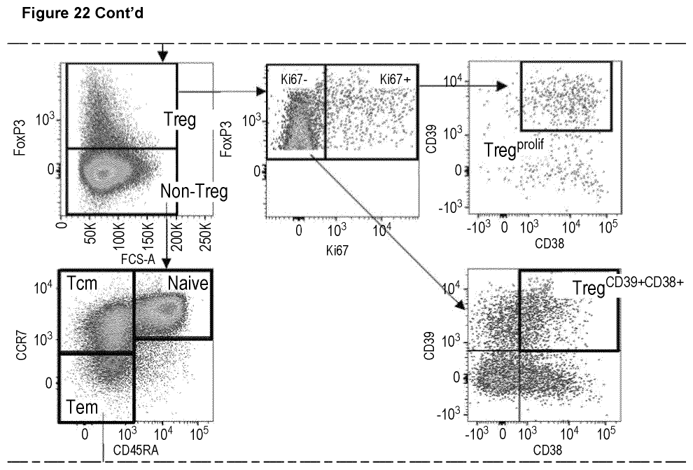

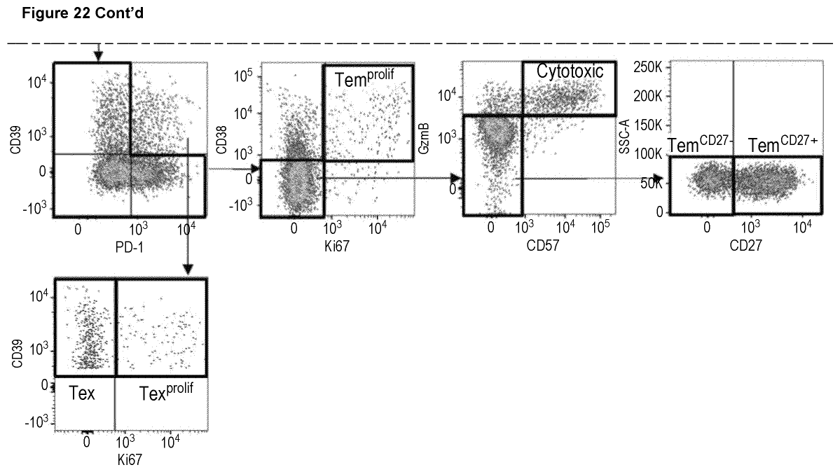

Alternative manual gating methods using a different panel of biomarkers are shown in FIG. 20 and FIG. 22, where CD8 T cells and CD4 T cells (including T regulatory subsets) are analysed separately. These are used for both the pre-invasive and the ASCENT data (see below). In both cases, manual gating frequencies can be used to validate computational clustering frequencies and may be used interchangeably.

### Example 2

The inventors performed further analysis of the data obtained in Example 1 to determine key biomarkers which are to form the inventive panel of biomarkers. A part of this analysis used the biomarkers Ki67 and CD39 to manually gate the clusters of total T cells isolated from a blood sample using CD3. Surprisingly, the expression of these two biomarkers were showed significantly differences between high-grade and low-grade disease. The inventors were able to show that subjects can be separated into high-grade or low-grade disease based on detection of Ki67 and CD39 expression on T cells. FIG. 1. This provides a simple and powerful tool requiring detection of expression of Ki67 and CD39. Based on the expression of Ki67 and CD39 biomarkers, a subject can be identified as being at risk for having a progressing or high-grade pre-invasive lesion or nodule or having a solid tumour. The analysis is based on a subject having more Ki67 and CD39 expressing T cells compared with the T cells in which expression of one or both of Ki67 CD39 is not detected.

### Example 3

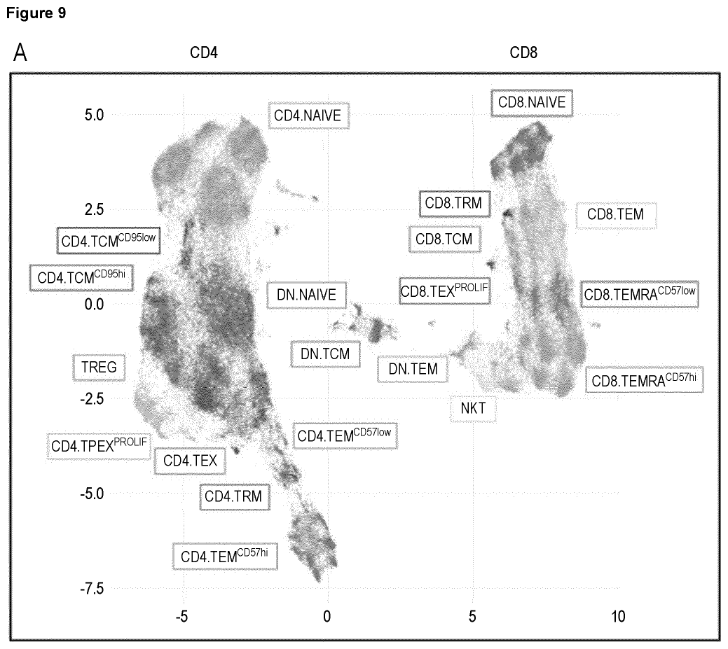

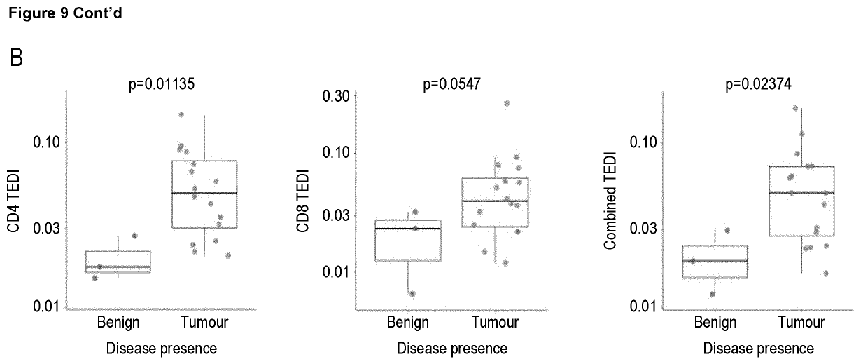

To assess if the methodology demonstrated in Examples 1 and 2 could be used in a cancer type with a different mutational landscape, blinded PBMC samples from patients with benign and malignant renal masses were assessed. T cell subsets were clustered and derived TEDI scores by deriving the ratio of exhausted:resting T cell clusters. FIG. 9. The results show an increase in systemic T cell differentiation in malignant (n=16 samples from 10 patients) vs benign (n=3 samples from 3 patients) disease. A) UMAP of FlowSOM defined T cell clusters from PBMC of all samples stained with 31 biomarkers and analysed by spectral cytometry. 5000 live CD3+ events per samples were down-sampled for analysis. B) The ratio of progenitor vs exhausted CD4 (left), CD8 (centre) or combined (right) T cell subsets. P values from one-tailed, unpaired Wilcoxin test. FIG. 9 shows that the ratio of activated/exhausted:naïve/resting T cells is increased in the blood of patients with renal cancer vs patients with benign disease. These results replicated the flow cytometry findings from pre-LUSC data suggesting broad utility of systemic T cell differentiation in multi-cancer early detection that can be used in all solid tumours from a variety of cancers.

### Example 4

The findings relating to skewing of T cell differentiation in the blood from pre-invasive patients were validated in a larger cohort of patients with early-stage, established non-small cell lung cancer (NSCLC).

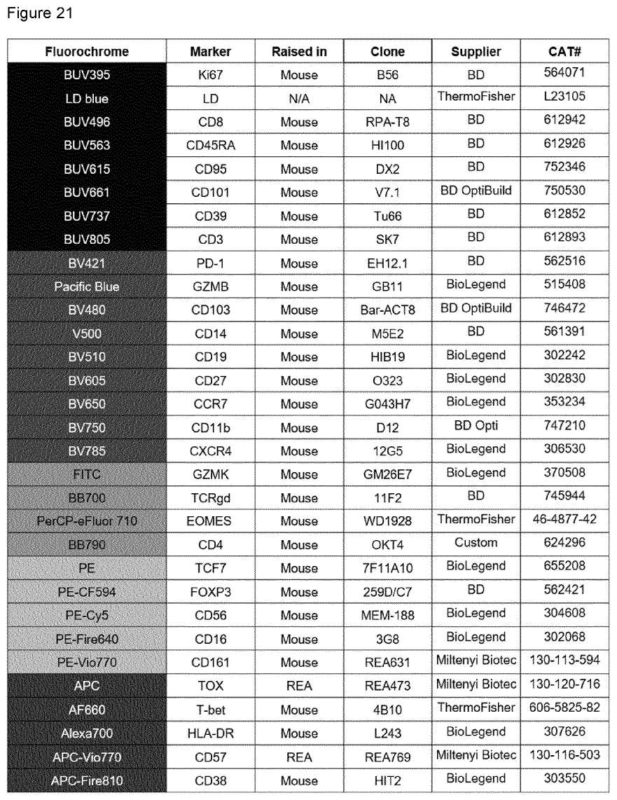

ASCENT is an observational study of patients with screen-detected lung cancer through the SUMMIT longitudinal surveillance study via LDCT scans. Blood (and tissue) samples are collected from surgery at the point of resection. For the ASCENT flow analysis, 86 patients were included with 57 NSCLC (35 LUAD (Lung adenocarcinoma)+22 LUSC (Lung squamous cell carcinoma) and 29 Healthy. Healthy age-matched blood samples were collected from patients going in for orthopaedic surgery, with no active infections or auto-immune conditions. ˜90% of NSCLC samples used in ASCENT analysis are Stage I (51 Stage I, 4 Stage II, 2 Stage III). The flow cytometry panel is shown in FIG. 21. Samples were acquired on a ID7000 Spectral Cell Analyser from Sony Biotechnology.

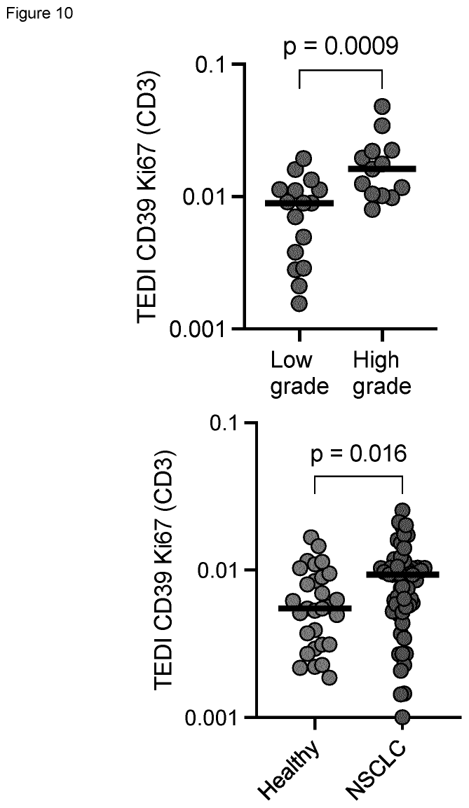

As shown in FIG. 10—Bottom), in early stage established disease, a higher ratio of activated:resting T cells was observed as was observed in pre-invasive disease. This is demonstrated by the TEDI CD39 Ki67 (CD3), where NSCLC patients have a higher proportion of CD3 CD39+Ki67+ cells than healthy. A similar plot with healthy patients versus LUSC gave a p-value of 0.14.

As shown in FIGS. 11A and 11B, when combining pre-invasive and NSCLC data, an increase in the TEDI CD39 Ki67 ratio is observed from healthy to low-grade to high-grade pre-invasive samples. The signal peaks at high-grade pre-invasive samples, dropping off at the lung cancer stage. This suggests the high-grade pre-invasive stage has the potential to be the most immunogenic and is therefore the optimum time to detect malignancy in the blood.

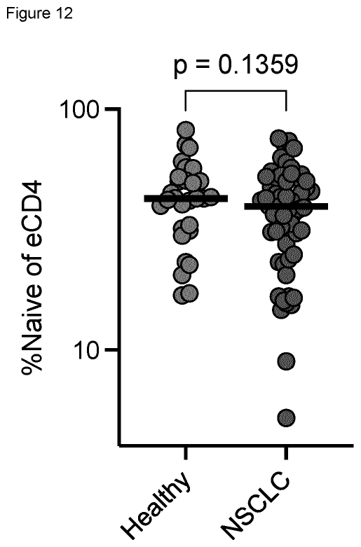

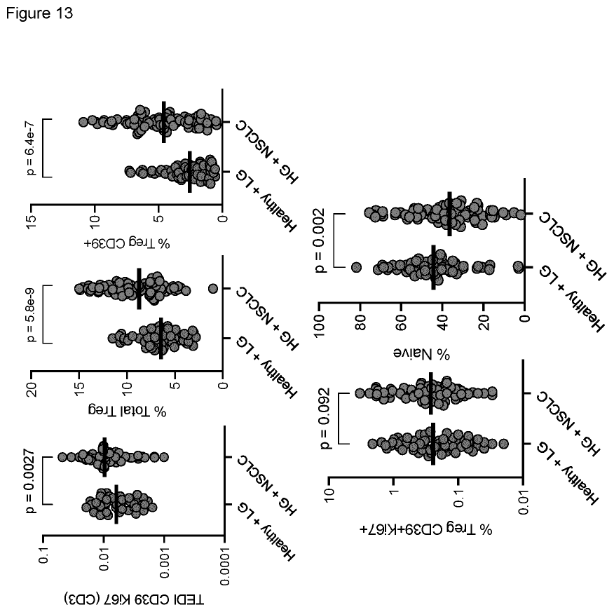

As well as lower frequency of CD39−Ki67− T cells in lung cancer patients, we also observe a trend of lower frequencies of naïve CD4+ T cells in NSCLC patients compared to healthy (p=0.1), similar to what was previously observed in the pre-invasive setting (see FIG. 12). When combining healthy and low grade (n=66) and combining high grade and NSCLC (n=88), there is still a significantly higher CD3 TEDI score in the disease/high-risk group compared with the healthy/low-risk group, coupled with a decrease in naïve cells in the CD4 T cell compartment (see FIG. 13).

### Example 5

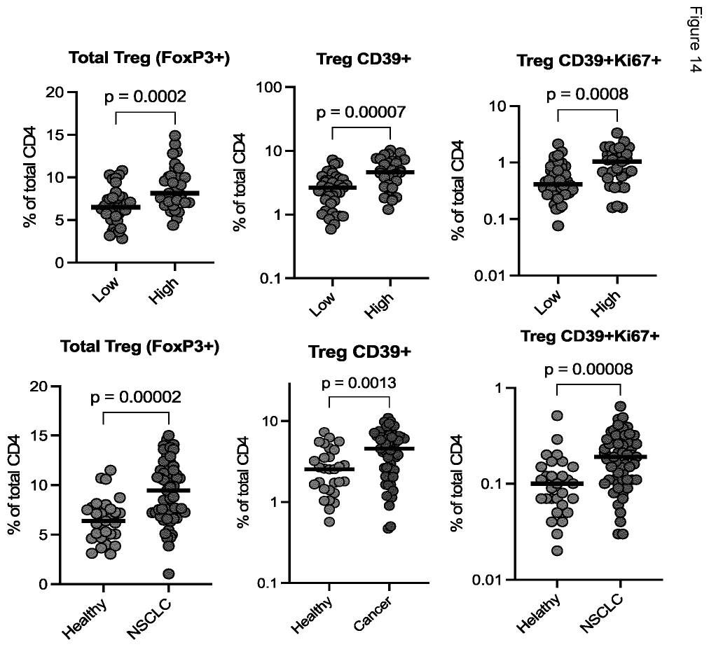

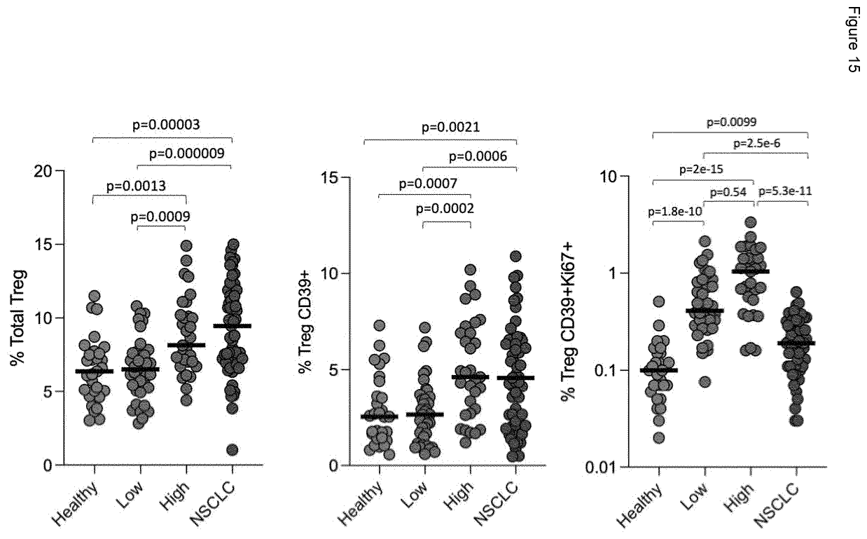

In further analysis of the pre-invasive flow cytometry data, the present inventors noted the importance of regulatory T cells (Tregs) expressing the biomarker FoxP3. Proliferating Tregs may also express the activation/proliferation markers CD39 and Ki67. In both pre-invasive and early-stage lung cancer, there was a significant increase in Proliferating CD39+Ki67+ Tregs in high-grade/lung cancer when compared with low-grade/healthy respectively. This is in accordance with more activated/memory cells supporting the finding that T cell differentiation is skewed during pre-invasive and invasive lung cancer, wherein patients having a higher risk of having a progressing/high-grade lesion/nodule/tumour to have higher frequencies of more activated/effort/regulatory T cells. This data is shown in FIG. 14. Looking at the pre-invasive and ASCENT data together, there is an increase from healthy to low grade and to high grade pre-invasive samples, with the signal from activated/proliferating Tregs peaking at high grade (see FIGS. 15).

When combining healthy and low-grade (n=66), and high-grade pre-invasive samples and NSCLC (n=88), there are also higher frequencies of total Tregs, CD39+ Tregs and CD39+Ki67+ Tregs (see FIG. 13).

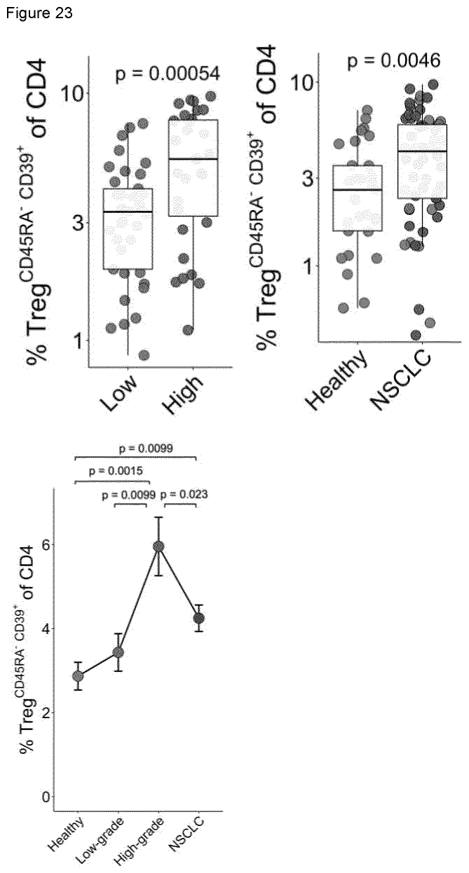

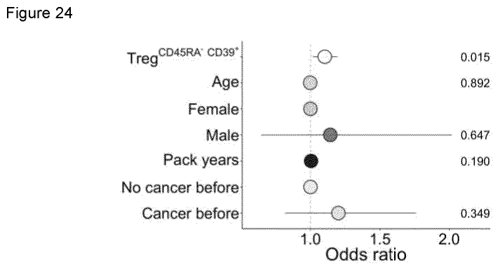

Tregs (i.e., CD4+ T cells expressing the biomarker FoxP3) also expressing the biomarker CD39 (CD39+) in the absence of the biomarker CD45RA (CD45RA−) were also significantly enriched in CD4+ cells in high-grade/early-stage lung cancer when compared with low-grade/healthy respectively also peaking in high pre-invasive samples (see FIG. 23).

### Example 6

It was also found that significant enrichment of Tem.Prolif.CD39hi cells (prolif=Ki67+) was observed in high grade pre-invasive lesions or nodules in live CD8 cells (CD3+CD8+ cells). These cells contain the presence of CD39 and Ki67 biomarkers, as well as having negative expression of CD45RA, CCR7 and PD1.

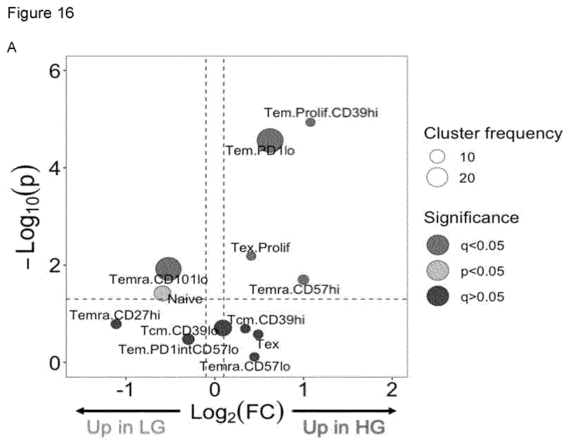

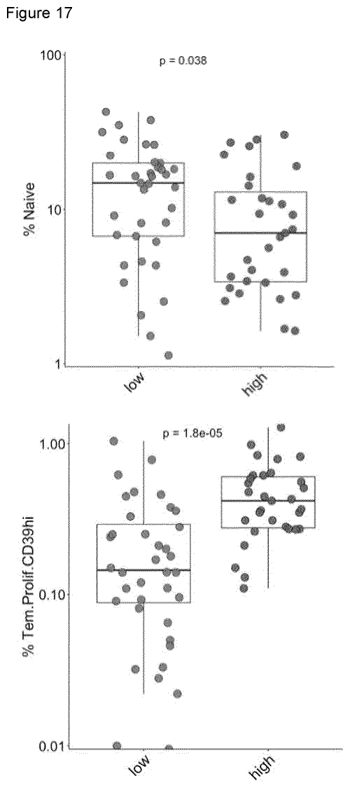

For the pre-invasive (PID) data, flow cytometry analysis is of 68 samples (31 high grade, 37 low grade) which passed the QC for having >3000 Live CD3+ and CD8+ cells used for analysis. FIG. 16 shows a volcano plot of populations determined by manual gating on flow cytometry that are significantly enriched in high grade pre-invasive lesions or nodules or low grade pre-invasive lesions or nodules. Significant enrichment of CD3+CD8+ Tem.Prolif.CD39hi cells (prolif=Ki67+) was observed in high grade pre-invasive lesions or nodules. FIG. 17 shows box plots for Top) naïve and Bottom) the above-mentioned Tem.Prolif.CD39hi populations (right) at sample level.

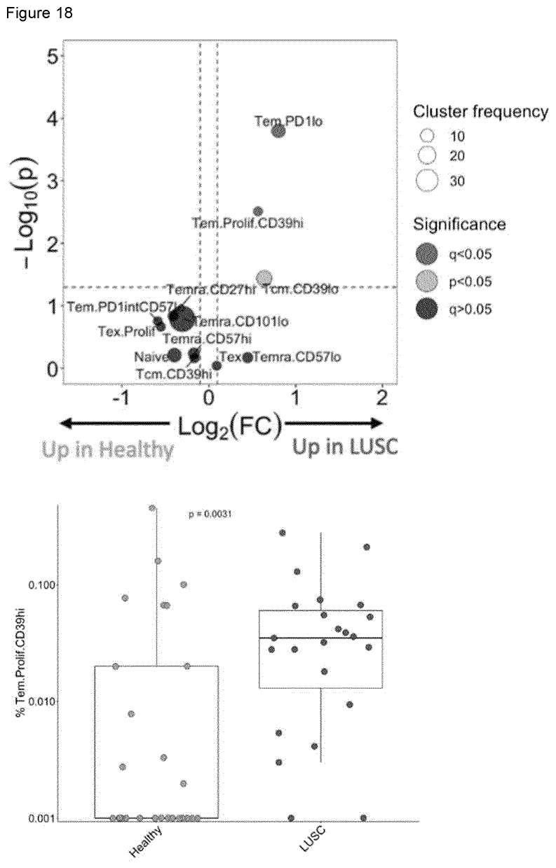

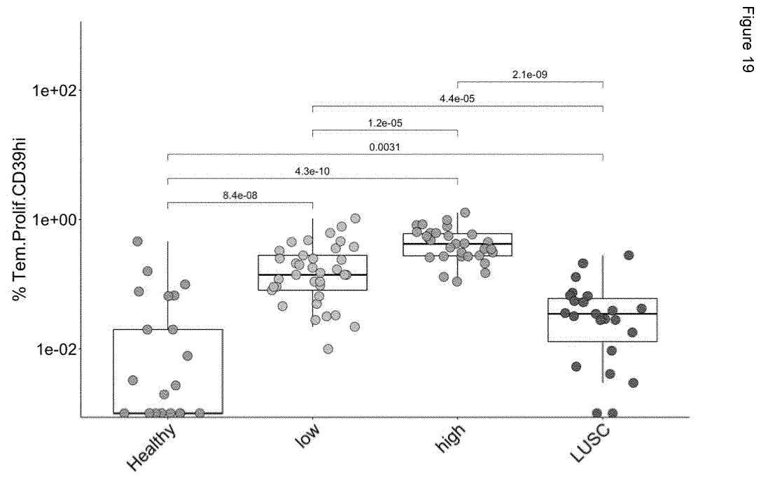

For the ASCENT data, the flow cytometry analysis is of 49 samples (27 Healthy and 22 LUSC) which passed the QC for having >1500 Live CD3+CD8+ cells were used for analysis. FIG. 18 (Top) shows a volcano plot of populations determined by manual gating that are significantly enriched in LUSC. FIG. 18 (Bottom) shows a box plot for the Tem.Prolif.CD39hi population which is significantly enriched in LUSC (Lung squamous cell carcinoma).

FIG. 19 shows the combined box plot values from both the PID (pre-invasive data) and ASCENT flow cytometry analysis to display the change in Tem.Prolif.CD39hi population and FIG. 20 shows the flow cytometry gating strategy used in this Example.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### Materials and Methods

### High-Dimensional Clustering

Clustering of flow cytometry data was completed following a modified pipeline from Nowicka et al [https://f1000research.com/articles/6-748]. FCS files underwent quality control of signal acquisition, assessed by the FlowAI package (v1.24) [https://academic.oup.com/bioinformatics/article/32/16/2473/2240408?login=false]. The arcsinh transformation was applied to the data, using the prepData function from the CATALYST package (v1.18.1) [https://www.ncbi.nlm.nih.gov/pmc/articles/PMC5981006/], with the cofactor set to 150 for standard fluorescence flow cytometry (PID cohort) and to 550 for spectral flow cytometry (ASCENT cohort). Any markers with a poor contribution to phenotypic variance were excluded pre-clustering. These were determined using the PCA-based non-redundancy score (NRS) as described in the Nowicka et al [https://f1000research.com/articles/6-748]. pipeline. CD103, Tim-3, TCF-7, CXCR4, TIGIT, and CD4 were excluded from clustering due to poor marker separation in the PID cohort, as well as the ASCENT cohort for consistency between panels (CD4 excluded as FCS files only included CD4⁺ T cells).

Data was clustered using the FlowSOM package (v2.2.0) [https://onlinelibrary.wiley.com/doi/full/10.1002/cyto.a.22625] onto a 10×10 node square self-organising map (SOM). Nodes were clustered by the ConsensusCenterPlus package (v1.58.0) [https://www.ncbi.nlm.nih.gov/pmc/articles/PMC2881355/], as described by Nowicka et al [https://f1000research.com/articles/6-748]. The UMAP algorithm was applied as a dimension reduction analysis to characterise the phenotypic relevance between individual clusters. The resulting clusters were manually merged based on marker expression similarity, occupation of space on the UMAP, and previously defined T cell states.

### Manual Gating

Subsets resolved from high-dimensional clustering were manually identified by conventional biaxial gating to ensure validity of clusters. PBMCs with ≥x viable T cells were analysed (PID cohort≥5000 CD4; ASCENT cohort≥4000 CD4). Manual gating analysis was carried out on FlowJo v10.8.1. Populations gated are shown on a concatenated file of all samples from one batch. Frequencies from total CD4 can be used to validate cluster significance from the high-dimensional clustering pipeline analysis

### Statistical Analysis

All statistical tests were performed in R (version 1.4.1106). Tests involving differences between groups were done using a ‘wilcox.test’ using unpaired filters, or a linear mixed effects model from the Ime4 (v1.1-30) [https://www.jstatsoft.org/article/view/v067i01] package. Details of the statistical test used are typically outlined in the corresponding figure legends. When required, p-values were adjusted using Benjamini-Hochberg (BH) p-value correction [doi:10.1214/193940307000000158], using the ‘p.adjust’ function.

Correlation analyses completed using the ‘cor.test’ function, with the method set to ‘Spearman’ and the alternative ‘two.sided’. Logistic regression models to account for potentially confounding variables were completed using the ‘glm’ function. Coefficients were exponentiated to get odds ratios with 95% confidence intervals. Significance was determined with a Wald's test, calculated by taking the cumulative probability associated with the absolute value of the Wald test statistic. All Wald's test p-values are two-tailed. All graphical presentation completed using the ggplot2 package (v3.3.6) [H. Wickham. ggplot2: Elegant Graphics for Data Analysis. Springer-Verlag New York, 2016.].

The present disclosure may be described by one or more of the following numbered paragraphs:
1. A method for determining whether a subject is at risk for having a progressing or high-grade pre-invasive lesion, nodule or small mass, or having a solid malignant tumour, the method comprising:
determining a ratio of activated and/or exhausted T cells:naïve and/or resting T cells in a sample of blood obtained from the subject,
wherein the determining comprises analysing T cells using cytometry to detect the presence or absence of a panel of biomarkers comprising Ki67 and CD39.
2. The method of paragraph 1, wherein the subject is at risk if
the ratio of activated and/or exhausted T cells:naïve and/or resting T cells is equal to or greater than
a ratio of activated and/or exhausted T cells:naïve and/or resting T cells of a comparison subject, or
an average ratio of activated and/or exhausted T cells:naïve and/or resting T cells of a plurality of comparison subjects.
3. A method for determining whether a subject is at risk for having a progressing or high-grade pre-invasive lesion, nodule or small mass, or having a solid malignant tumour, the method comprising:
determining a ratio of activated and/or exhausted T cells:T cells which are not activated and/or exhausted T cells in a sample of blood obtained from the subject,
wherein the determining comprises analysing T cells using cytometry to detect the presence or absence of a panel of biomarkers comprising Ki67 and CD39.
4. The method of paragraph 3, wherein the subject is at risk if
the ratio of activated and/or exhausted T cells:T cells which are not activated and/or exhausted is equal to or greater than
a ratio of activated and/or exhausted T cells:T cells which are not activated and/or exhausted of a comparison subject, or
an average ratio of activated and/or exhausted T cells:T cells which are not activated and/or exhausted of a plurality of comparison subjects.
5. The method of paragraph 2 or 4 wherein, a comparison subject is selected from:
a) a subject known to have a progressing or high-grade pre-invasive lesion, nodule or small mass or an established solid malignant tumour,
b) the subject at a different time point.
6. The method of paragraph 2 or 4 wherein, a plurality of comparison subjects is selected from:
a) a plurality of subjects known to have a progressing or high-grade pre-invasive lesion, nodule or small mass or an established solid malignant tumour,
b) a plurality of healthy subjects, or
c) a plurality of subjects of the general population.
7. The method of any one of the preceding paragraphs, wherein an activated and/or exhausted T cell expresses CD39 and Ki67 (CD39+ and Ki67+ T cells).
8. The method of any one of the preceding paragraphs, wherein naïve and/or resting T cells do not express CD39 or Ki67 (CD39− and Ki67− T cells).
9. The method of any one of the preceding paragraphs, wherein T cells which are not activated and/or exhausted do not express one or both of CD39 or Ki67 (CD39− and Ki67+ T cells, CD39+ and Ki67− T cells, or CD39− and Ki67− T cells).
10. The method of any one of the preceding paragraphs wherein the panel of biomarkers further comprises one or more biomarkers selected from CD45RA, CCR7, PD-1, CD57 or CD38.
11. The method of any one of the preceding paragraphs wherein the panel of biomarkers further comprises one or more biomarkers selected from CD3, CD4 or CD8.
12. The method of any one of the preceding paragraphs wherein the analysis comprises use of a viability dye.
13. The method of any one of paragraphs 1 to 10, wherein the panel of biomarkers further comprises CD45RA, or CCR7, or CD45RA and CCR7.
14. The method of any one of paragraphs 1 to 10 wherein, the panel of biomarkers further comprises CD45RA, CCR7, and PD-1.
15. The method of any one of paragraphs 1 to 10, wherein the panel of biomarkers further comprises CD45RA, CCR7, PD-1 and CD57.
16. The method of any one of paragraphs 1 to 10, wherein the panel of biomarkers further comprises CD45RA, CCR7, PD-1, CD57 and CD38.
17. The method of any one of paragraphs 1 to 10, wherein the panel of biomarkers further comprises CD45RA, CCR7, CD57, and CD38.
18. The method of any one of paragraphs 1 to 10, wherein the panel of biomarkers further comprises CD45RA, PD-1 and CD57.
19. The method of any one of the preceding paragraphs, wherein the cytometry comprises one or more of flow cytometry, spectral cytometry or mass cytometry, optionally the cytometry comprises flow cytometry.
20. A kit comprising a set of lyophilised antibodies or a fragment thereof, comprising antibodies binding to CD39 and Ki67.
21. The kit according to paragraph 20, wherein the set of lyophilised antibodies or fragment thereof comprises antibodies binding to the further biomarkers as defined in any one of paragraphs 10 to 18.
22. Use of a kit according to paragraph 20 or 21 in a method for determining whether a subject is at risk for having a progressing or high-grade pre-invasive lesion, nodule or small mass, or having a solid malignant tumour.
23. A device comprising:
(i) means for receiving a sample of blood, and
(ii) a set of lyophilised antibodies or a fragment thereof, comprising antibodies binding to CD39 and Ki67.
24. A method of treating a subject determined to be at risk for having a progressing or high-grade pre-invasive lesion, nodule or small mass, or having a solid malignant tumour, wherein the method comprises:
   determining whether a subject is at risk for having a progressing or high-grade pre-invasive lesion, nodule or small mass, or having a solid malignant tumour, the method comprising:
determining a ratio of activated and/or exhausted T cells:naïve and/or resting T cells in a sample of blood obtained from the subject,
wherein the determining comprises analysing T cells using cytometry to detect the presence or absence of a panel of biomarkers comprising Ki67 and CD39, and
wherein the subject is at risk if
the ratio of activated and/or exhausted T cells:naïve and/or resting T cells is equal to or greater than
a ratio of activated and/or exhausted T cells:naïve and/or resting T cells of a comparison subject, or
an average ratio of activated and/or exhausted T cells:naïve and/or resting T cells of a plurality of comparison subjects,
and
   providing treatment to said subject, if said subject is determined to be at risk.
25. A method of treating a subject determined to be at risk for having a progressing or high-grade pre-invasive lesion, nodule or small mass, or having a solid malignant tumour, wherein the method comprises:
   determining whether a subject is at risk for having a progressing or high-grade pre-invasive lesion, nodule or small mass, or having a solid malignant tumour, the method comprising:
determining a ratio of activated and/or exhausted T cells:T cells which are not activated and/or exhausted T cells in a sample of blood obtained from the subject,
wherein the determining comprises analysing T cells using cytometry to detect the presence or absence of a panel of biomarkers comprising Ki67 and CD39, and
wherein the subject is at risk if
the ratio of activated and/or exhausted T cells:T cells which are not activated and/or exhausted is equal to or greater than
a ratio of activated and/or exhausted T cells:T cells which are not activated and/or exhausted of a comparison subject, or
an average ratio of activated and/or exhausted T cells:T cells which are not activated and/or exhausted of a plurality of comparison subjects,
and
   providing treatment to said subject, if said subject is determined to be at risk.

1-29. (canceled)

30. A method of treating a subject, wherein the method comprises:
determining whether the subject is at risk for having a progressing or high-grade pre-invasive lesion, nodule or small mass, or having a solid malignant tumour by:
(i) determining a ratio of activated and/or exhausted T cells:naïve and/or resting T cells in a sample of blood obtained from the subject, wherein the determining comprises analysing T cells using cytometry to detect the presence or absence of a panel of biomarkers comprising Ki67 and CD39, and wherein the subject is at risk if the ratio of activated and/or exhausted T cells:naïve and/or resting T cells is equal to or greater than
a ratio of activated and/or exhausted T cells:naïve and/or resting T cells of a comparison subject, or
an average ratio of activated and/or exhausted T cells:naïve and/or resting T cells of a plurality of comparison subjects;
(ii) determining a ratio of activated and/or exhausted T cells:T cells which are not activated and/or exhausted T cells in a sample of blood obtained from the subject, wherein the determining comprises analysing T cells using cytometry to detect the presence or absence of a panel of biomarkers comprising Ki67 and CD39, and wherein the subject is at risk if the ratio of activated and/or exhausted T cells:T cells which are not activated and/or exhausted is equal to or greater than
a ratio of activated and/or exhausted T cells:T cells which are not activated and/or exhausted of a comparison subiect, or
an average ratio of activated and/or exhausted T cells:T cells which are not activated and/or exhausted of a plurality of comparison subjects; or
(iii) determining a proportion of activated and/or exhausted T cells as a percentage of T cells in a sample of blood of the subject, wherein the determining comprises analysing T cells using cytometry to detect either (a) the presence of Ki67 and CD39 and/or (b) the presence of FoxP3, wherein the activated and/or exhausted T cells are CD4 T cells, and wherein the subject is at risk if
the proportion of activated and/or exhausted T cells is higher than the proportion of activated and/or exhausted T cells in a comparison subject, or
the proportion of activated and/or exhausted T cells is higher than the average proportion of activated and/or exhausted T cells in a plurality of comparison subjects;
determining the subiect is at risk for having a progressing or high-grade pre-invasive lesion, nodule or small mass, or having a solid malignant tumour; and
providing treatment to the subject based on the determination that the subject is at risk.

31. The method of claim 30, wherein the solid malignant tumour is a stage I solid malignant tumour.

32. The method of claim 30, wherein the subject is determined to be at risk for having a progressing or high-grade pre-invasive lesion, nodule or small mass.

33. The method of claim 30, wherein the method comprises (iii) determining the proportion of activated and/or exhausted T cells as a percentage of T cells in the sample of blood of the subject.

34. The method of claim 33, wherein the determining comprises analysing T cells using cytometry to detect the presence of Ki67 and CD39.

35. The method of claim 33, wherein the determining comprises analysing T cells using cytometry to detect the presence of FoxP3, wherein the activated and/or exhausted T cells are CD4 T cells.

36. The method of claim 30, wherein the method comprises (i) determining the ratio of activated and/or exhausted T cells:naïve and/or resting T cells in the sample of blood obtained from the subject.

37. The method of claim 30, wherein the method comprises (ii) determining the ratio of activated and/or exhausted T cells:T cells which are not activated and/or exhausted T cells in the sample of blood obtained from the subject.

38. The method of claim 30, wherein the comparison subject is selected from:
a) a subject known to have a progressing or high-grade pre-invasive lesion, nodule or small mass or an established solid malignant tumour,
b) the subject at a different time point,
c) a plurality of subjects known to have a progressing or high-grade pre-invasive lesion, nodule or small mass or an established solid malignant tumour,
d) a plurality of healthy subjects, or
e) a plurality of subjects of the general population.

39. The method of claim 30, wherein the activated and/or exhausted T cell expresses CD39 and Ki67.

40. The method of claim 36, wherein the naïve and/or resting T cells do not express CD39 or Ki67.

41. The method of claim 37, wherein the T cells which are not activated and/or exhausted do not express one or both of CD39 or Ki67.

42. The method of claim 30, wherein the panel of biomarkers of (i) and/or (ii) further comprises:
a) one or more biomarkers selected from CD45RA, CCR7, PD-1, CD57, or CD38;
b) one or more biomarkers selected from CD3, CD4, or CD8;
c) CD45RA, CCR7, or CD45RA and CCR7;
d) CD45RA, CCR7, and PD-1;
e) CD45RA, CCR7, PD-1, and CD57;
f) CD45RA, CCR7, PD-1, CD57, and CD38;
g) CD45RA, CCR7, CD57, and CD38; or
h) CD45RA, PD-1, and CD57.

43. The method of claim 35, wherein the activated and/or exhausted T cell expresses FoxP3 in combination with CD39.

44. The method of claim 43, wherein the activated and/or exhausted T cell expresses (i) FoxP3 in combination with CD39 and Ki67, or (ii) FoxP3 in combination with CD39 and does not express CD45RA.

45. The method of claim 30, wherein the cytometry comprises one or more of flow cytometry, spectral cytometry, or mass cytometry

46. The method of claim 30, wherein the cytometry comprises flow cytometry.

47. The method of claim 30, wherein analysing the T cells comprises use of a viability dye.

48. The method of claim 30, wherein the treating comprises administering an anti-cancer therapeutic.

49. The method of claim 30, wherein the treating comprises electrocautery, argon plasma coagulation (APC), cryotherapy, or photodynamic therapy (PDT).
